(19) United States
(12) Patent Application Publication
(10) Pub. No.: US 2021/0348237 A1
(43) Pub. Date: Nov. 11, 2021

(54) METHODS AND COMPOSITIONS FOR AIDING IN THE DETECTION OF LUNG CANCER

(71) Applicant: 20/20 GeneSystems, Rockville, MD (US)

(72) Inventor: William James, Rockville, MD (US)

(73) Assignee: 20/20 GeneSystems, Rockville, MD (US)

(21) Appl. No.: 17/182,142

(22) Filed: Feb. 22, 2021

Related U.S. Application Data
(63) Continuation of application No. 14/483,503, filed on Sep. 11, 2014, now abandoned.
(60) Provisional application No. 61/876,740, filed on Sep. 11, 2013.

(51) Int. Cl. C12Q 1/6886; G01N 33/574

(52) U.S. Cl. CPC C12Q 1/6886; G01N 33/57423; G01N 2800/50; C12Q 2600/178; G01N 33/57484

(57) ABSTRACT

A lung cancer biomarker panel comprising an microRNA (miRNA) lung cancer biomarker and at least one additional lung cancer biomarker selected from a tumor protein (TP) lung cancer biomarker and/or a autoantibody (AAB) lung cancer biomarker is provided herein and methods for screening patients for lung cancer. The present lung cancer biomarker panel provides an improvement in sensitivity and diagnostic accuracy for lung cancer as compared to a lung cancer biomarker panel without the miRNA biomarkers.

### CROSS REFERENCE TO RELATED APPLICATIONS

This application claims benefit under 35 U.S.C. 1.19(e) of and priority to U.S. Provisional patent application Ser. No. 61/876,740 filed Sep. 11, 2013, the content of which hereby is incorporated by reference in entirety.

### FIELD

The disclosure relates to lung cancer biomarker panels and screening methods for the presence of cancer in an asymptomatic human subject.

### BACKGROUND

Lung cancer is by far the leading cause of cancer deaths in North America and most of the world killing more people than the next three most lethal cancers combined, namely breast, prostate, and colorectal cancer. Lung cancer results in over 156,000 deaths per year in the United States alone (American Cancer Society. Cancer Facts & Figures 2011. Atlanta: American Cancer Society; 2011). Tobacco use has been identified as a primary causal factor for lung cancer and is thought to account for some 90% of cases. Thus, individuals over 50 years of age with a smoking history of greater than 20 pack-years have a 1 in 7 lifetime risk of developing the disease. Lung cancer is a relatively silent disease displaying few if any specific symptoms until it reaches the later more advanced stages. Therefore most patients are not diagnosed until their cancer has metastasized beyond the lung and they are no longer treatable by surgery alone. Thus, while the best way to prevent lung cancer is likely tobacco avoidance or cessation, for many current and former smokers, the transforming, cancer-causing event has already occurred and even though the cancer is not yet manifest, the damage is already done. Thus, perhaps the most effective means of reducing lung cancer mortality today is early stage detection when the tumor is still localized and amenable to surgery with intent to cure.

The importance of early detection was recently demonstrated in a large 7-year clinical study, the National Lung Cancer Screening Trial (NLST), which compared chest x-ray and chest CT scanning as potential modalities for the early detection of lung cancer (National Lung Screening Trial Research Team, Aberle D R, Adams A M, Berg C D, Black W C, Clapp J D, Fagerstrom R M, Gareen I F, Gatsonis C, Marcus P M, Sicks J D. Reduced lung-cancer mortality with low-dose computed tomographic screening. N Engl J Med. 2011 Aug. 4; 365(5):395-409). The trial concluded that the use of chest CT scans to screen the at-risk population identified significantly more early stage lung cancers than chest x-ray and resulted in a 20% overall reduction in disease mortality. This study has clearly indicated that identifying lung cancer early can save lives. Unfortunately, the broad application of CT scanning as a screening method for lung cancer is not without problems. The NLST design utilized a serial CT screening paradigm in which patients received a CT scan annually for only three years. Nearly 40% of the participants receiving the annual CT scan over 3 years had at least one positive screening result and 96.4% of these positive screening results were false positives. This very high rate of false positives can cause patient anxiety and a burden on the healthcare system, as the work-up following a positive finding on low-dose CT scans often includes advanced imaging and biopsies. Although CT scanning is an important tool for the early detection of lung cancer, more than two years after the NLST results were announced, very few patients at high risk for lung cancer due to smoking history have initiated a program of annual CT scans. This reluctance to undergo yearly CT scans is likely due to a number of factors including costs, perceived risks of radiation exposure especially by serial CT scans, the inconvenience or burden to asymptomatic patients of scheduling a separate diagnostics procedure at a radiology center, as well as concerns by physicians that the very high false positive rates of CT scanning as a standalone test will result in a significant number of unnecessary follow up diagnostic tests and invasive procedures.

While the overall lifetime risk for lung cancer amongst smokers is high, the chance that any individual smoker has cancer at a specific point in time is only on the order of 1.5-2.7% [Bach, P. B., et al., Screening for Lung Cancer*ACCP Evidence-Based Clinical Practice Guidelines (2nd Edition). CHEST Journal, 2007. 132(3_suppl): p. 69S-77S.]. Due to this low disease prevalence, a simple method to better identify which patients are at highest risk is necessary. The ideal method would be non-invasive, highly accurate and easily performed in the context of the standard work-up of the patient at a yearly physician visit with the standard blood work-up. Such a test needs to have at least a moderate level of sensitivity and be amenable to serial testing with a high level of patient compliance. The best format for such a test that meets all of these requirements is a simple blood test.

At present, there is still a need for clinically relevant markers for non-invasive detection of lung disease including cancer, monitoring response to therapy, or detecting lung cancer recurrence. It is also clear that such assays must be highly specific with reasonable sensitivity, and be readily available at a reasonable cost. Circulating biomarkers offer an alternative to imaging with the following advantages: 1) they are found in a minimally-invasive, easy to collect specimen type (blood or blood-derived fluids), 2) they can be monitored frequently over time in a subject to establish an accurate baseline, making it easy to detect changes over time, 3) they can be provided at a reasonably low cost, 4) they may limit the number of patients undergoing repeated expensive and potentially harmful CT scans, and/or 5) unlike CT scans, biomarkers may potentially distinguish indolent from more aggressive lung lesions (see, e.g., Greenberg and Lee, Opin Pulm Med, 13:249-55 (2007)).

Existing biomarker assays include several serum protein markers such as CEA (Okada et al., Ann Thorac Surg, 78:216-21 (2004)), CYFRA 21-1 (Schneider, Adv Clin Chem, 42:1-41 (2006)), CRP (Siemes et al., J Clin Oncol, 24:5216-22 (2006)), CA-125 (Schneider, 2006), and neuron-specific enolase and squamous cell carcinoma antigen (Siemes et al., 2006). Low sensitivity and specificity, with a significant number of false positive results due to benign pulmonary diseases have limited the application of these assays.

Circulating nucleic acids such as DNA and mRNA have also been evaluated as possible diagnostic markers for lung cancer. These studies are based on the observations that circulating nucleic acids show differential expression that is suggestive of cancer. (See, e.g., Bremnes et al., Lung Cancer, 49:1-12 (2005); Johnson et al., Cell, 120:635-47 (2005); Yanaihara et al., Cancer Cell, 9:189-98 (2006); Chen et al., Cell Res, 18:997-1006 (2008); Fabbri et al., Cancer J, 14:1-6 (2008); Garofalo et al., Oncogene, 27:3845-55 (2008); Mitchell et al., Proc Natl Acad Sci, 105:10513-8 (2008); Schickel et al., Oncogene, 27:5959-74 (2008); Weiss et al., Ann Oncol, 19:1053-9 (2008); and Yu et al., Cancer Cell, 13:48-57 (2008).) The origin of free DNA in circulation is not completely understood, but they are thought to represent the stable remaining fraction from damaged (apoptotic, necrotic) tumor cells (Jahr et al., Cancer Res, 61:1659-65 (2001); Bianchi, Placenta, 25 Suppl A:S93-S101 (2004)).

Micro-RNAs (miRNAs) are part of a large class of short, non-coding RNAs that regulate expression of genes. They interact with messenger RNA (mRNA) by specific binding in an anti-sense mode, thus either inducing mRNA degradation, or inhibiting mRNA translation into protein. Biological feed-back loops occur, in which there is reciprocal inhibition of miRNA and the target mRNAs. MiRNA expression profiles are associated with many malignancies including lung cancer, and in cancer cells specific oncogenes are regulated by certain miRNAs. This is a biological mechanism for control of gene expression at the mRNA level, in contrast to the functions of expressed cell signaling proteins, which can control both gene transcription and cell-cell interactions.

Several studies of mRNAs downregulated by miR-21 consistently identified target mRNAs encoding cell cycle checkpoints regulators, suggesting an important role for miR-21 in oncogenic RAS-induced cell proliferation (Markou, A., Y. Liang, and E. Lianidou, Prognostic, therapeutic and diagnostic potential of microRNAs in non-small cell lung cancer. Clinical Chemistry & Laboratory Medicine, 2011. 49(10): p. 1591-1603). In 2008 a number of labs published findings that miRNAs circulate in a highly stable, cell-free form in the blood, and can be detected in plasma, serum and sputum (Chen, X., et al., Characterization of microRNAs in serum: a novel class of biomarkers for diagnosis of cancer and other diseases. Cell Res, 2008. 18(10): p. 997-1006; Mitchell, P. S., et al., Circulating microRNAs as stable blood-based markers for cancer detection. Proceedings of the National Academy of Sciences, 2008. 105(30): p. 10513-10518).

We herein describe lung cancer biomarker panels comprising at least one miRNA lung cancer biomarker and, at least one additional tumor protein (TP) lung cancer biomarker and/or autoantibody (AAB) lung cancer biomarker to be used for lung cancer screening, wherein the lung cancer panel provides improved sensitivity, specificity and diagnostic accuracy for lung cancer.

These and other advantages of the present invention may be better understood by referring to the following description, accompanying drawings and claims. This description of an embodiment, set out below to enable one to practice an implementation of the invention, is not intended to limit the preferred embodiment, but to serve as a particular example thereof. Those skilled in the art should appreciate that they may readily use the conception and specific embodiments disclosed as a basis for modifying or designing other methods and systems for carrying out the same purposes of the present invention. Those skilled in the art should also realize that such equivalent assemblies do not depart from the spirit and scope of the invention in its broadest form.

### SUMMARY

The present disclosure provides processes for assessing the likelihood that a patient has lung cancer by measuring levels of lung cancer biomarkers in a sample from a patient. The measured lung cancer biomarkers comprise at least one microRNA (miRNA) lung cancer biomarker and at least one additional lung cancer biomarker selected from a tumor protein (TP) lung cancer biomarker and/or an autoantibody (AAB) lung cancer biomarker. A probability of cancer is then calculated from the measured lung cancer biomarkers, in aggregate, to determine the likelihood the patient has lung cancer. Inclusion of an miRNA lung cancer biomarker in the panel of measured biomarkers markedly increases the sensitivity, with a high specificity, for lung cancer.

In certain aspects the measured lung cancer biomarkers include miRNA and TP lung cancer biomarkers; miRNA and AAB lung cancer biomarkers; or miRNA, TP and AAB lung cancer biomarkers. The miRNA lung cancer biomarkers may be selected from Mir21, Mir126, Mir210 or Mir486. The TP and AAB lung cancer biomarkers may be selected from CEA, CA125, Cyfra 21-1, Pro-GRP, anti-NY-ESO-1, anti-p53, anti-Cyclin E2 and anti-MAPKAPK3.

### BRIEF DESCRIPTION OF THE FIGURES

The numerous advantages of the present invention may be better understood by those skilled in the art by reference to the accompanying figures in which:

FIG. 1 shows sensitivity (at 80% specificity) and area under the curve (AUC) values for each biomarker measured as determined by a receiver operator characteristic (ROC) curve analysis for lung cancer in table format of each of the 10 lung cancer biomarkers measured.

FIG. 2 shows sensitivity (at 80% specificity) for lung cancer in bar graph format of each of the 10 lung cancer biomarkers measured.

FIG. 3 shows the area under the curve (AUC) values for each biomarker measured as determined by a receiver operator characteristic (ROC) curve analysis for all lung cancer vs. all non-cancer samples.

FIG. 4 shows a table for the seven groups of lung cancer biomarkers that were measured of their respective sensitivity (at 80% specificity) and area under the curve (AUC) values as determined by a receiver operator characteristic (ROC) curve analysis for lung cancer.

FIG. 5 shows sensitivity (at 80% specificity) for lung cancer in bar graph format for each of the seven groups of lung cancer biomarkers from FIG. 4.

FIG. 6 shows the area under the curve (AUC) values for each of the seven groups from FIG. 4 as determined by a receiver operator characteristic (ROC) curve analysis for all lung cancer vs. all non-cancer samples. Group 5 (miRNA and TP biomarkers) and Group 7 (miRNA; TP and AAB biomarkers) each had an AUC value greater than 0.90.

FIG. 7 shows a comparison of receiver operator characteristic (ROC) curves for combination of biomarkers in Control Group 1 (miRNA biomarkers), Control Group 2 (TP biomarkers) and Test Group 6 (miRNA and TP biomarkers). Group 6 demonstrates an AUC value of 0.93.

FIG. 8 shows a comparison of receiver operator characteristic (ROC) curves for combination of biomarkers in Control Group 1 (miRNA biomarkers), Control Group 3 (AAB biomarkers) and Test Group 5 (miRNA and AAB biomarkers). Group 5 demonstrates an AUC value of 0.89.

FIG. 9 shows a comparison of receiver operator characteristic (ROC) curves for combination of biomarkers in Control Group 1 (miRNA biomarkers), Control Group 4 (TP and AAB biomarkers) and Test Group 7 (miRNA and TP and AAB biomarkers). Group 7 demonstrates an AUC value of 0.95.

FIG. 10 shows an example calculation with multiple logistic regression analysis for calculating the probability of cancer.

### DETAILED DESCRIPTION

A) Introduction

The present disclosure is based, in part, on the discovery that using microRNA (miRNA) lung cancer biomarkers in combination with tumor protein (TP) lung cancer biomarkers and/or autoantibody (AAB) lung cancer biomarkers, surprisingly increases the sensitivity and/or specificity and/or diagnostic accuracy for lung cancer. Provided herein are panels of lung cancer biomarkers comprising at least one miRNA lung cancer biomarker and at least one additional lung cancer biomarker from the group of TP or AAB lung cancer biomarkers. These panels are used in screening methods for determining the likelihood a patient has lung cancer with a high degree of diagnostic accuracy.

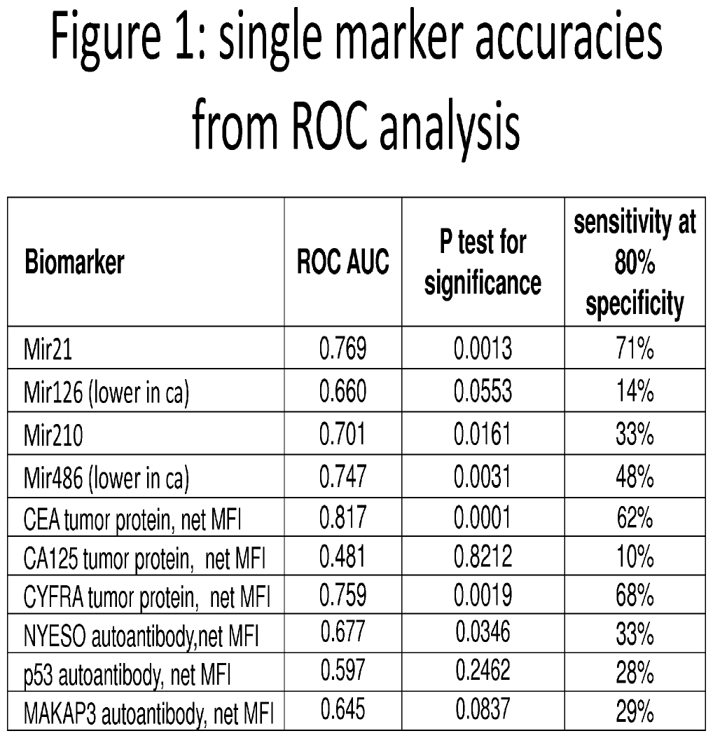

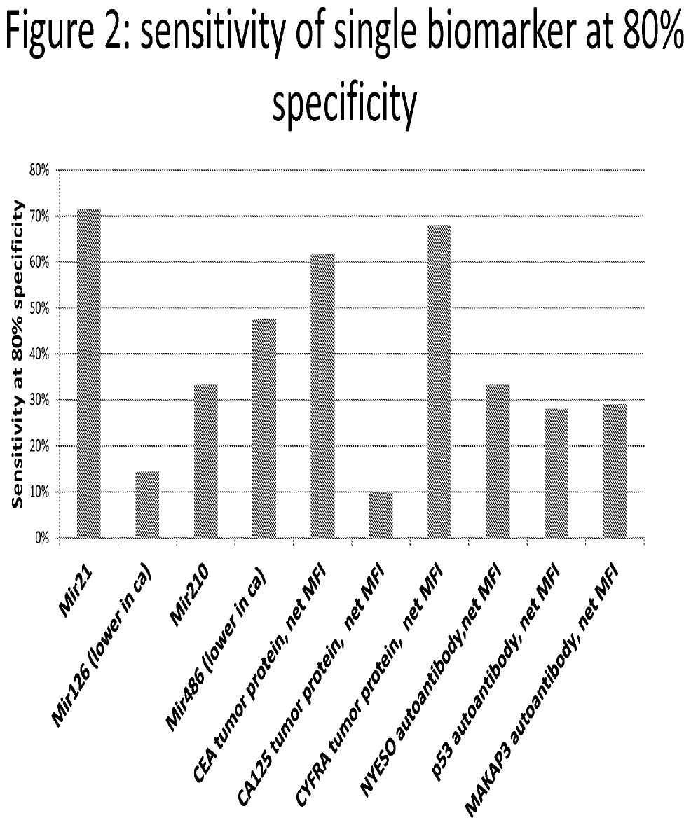

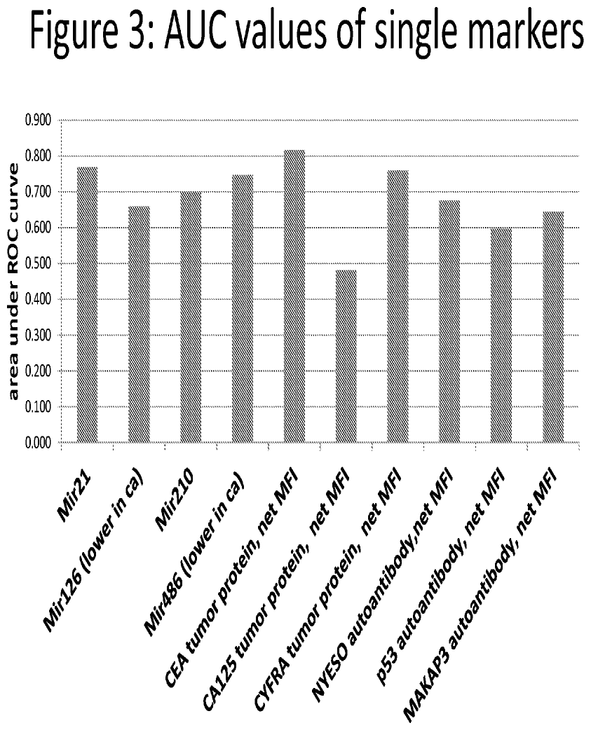

Samples from a cohort of 24 cases of stage 1a and Ib lung cancer, consisting of 13 adenocarcinomas and 11 squamous cell carcinomas, and 26 matched benign lung lesions were obtained from the Veterans Administration. See Table 1 in Example 1. The level of four miRNA, three TP and three AAB lung cancer biomarkers were measured from the samples (data not shown). The measured levels of the 10 lung cancer biomarkers were analyzed for AUC values; sensitivity and specificity for lung cancer. See FIG. 1. The sensitivity for each individual lung cancer biomarker, at 80% specificity, ranged from 10% to 71%. For the lung cancer biomarkers in the TP group, the sensitivity was 68%, 10% and 62%; for the lung cancer biomarkers in the AAB group, the sensitivity was 29%, 28% and 33%; and the sensitivity for the biomarkers in the miRNA group was 71%, 14%, 33% and 48%. See FIG. 2. The corresponding AUC values for these ten individual biomarkers are shown in FIGS. 1 and 3.

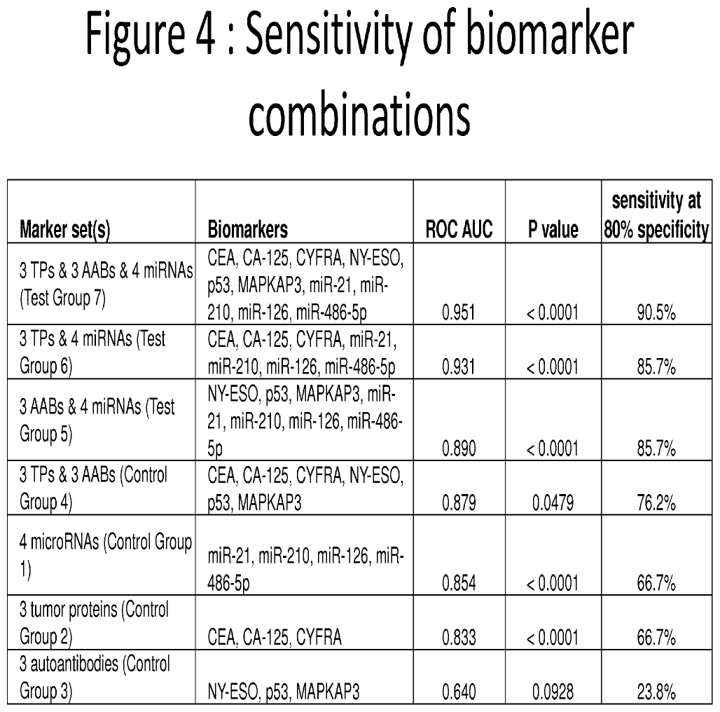

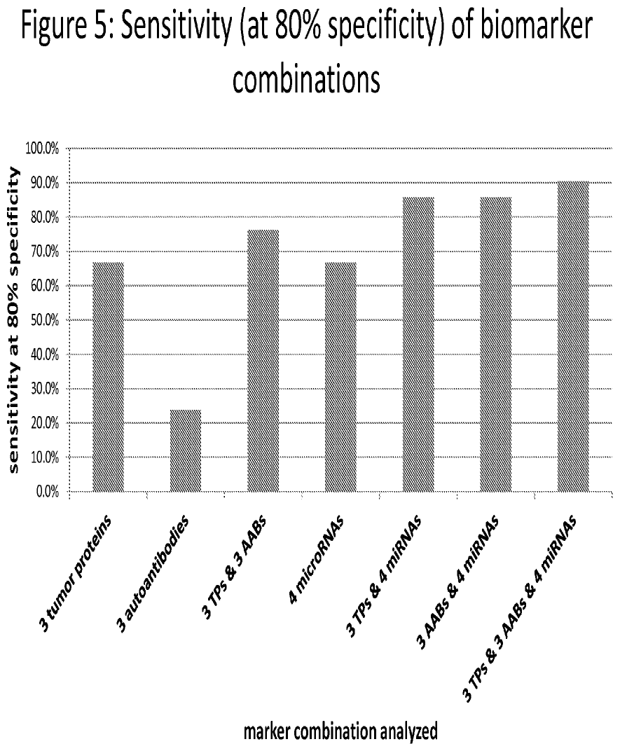

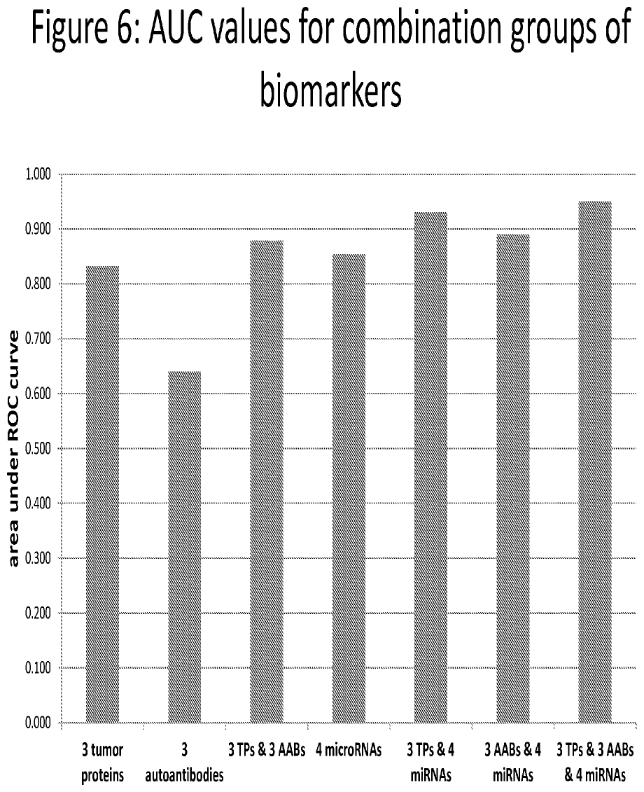

The ten (10) lung cancer biomarkers were analyzed as seven distinct groups of biomarkers—miRNA (Control group 1); TP (Control Group 2); AAB (Control Group 3); AAB and TP (Control Group 4); miRNA and AA(Test Group 5); miRNA and TP (Test Group 6); miRNA and TP and AAB (Test Group 7). See FIG. 4. The sensitivity for each group, at 80% specificity, demonstrates that each of Test Group 5, 6 and 7 had a significant increase in sensitivity as compared to their respective Control Groups. See FIG. 5. The corresponding AUC values for these seven groups of lung cancer biomarkers are shown in FIGS. 4 and 6.

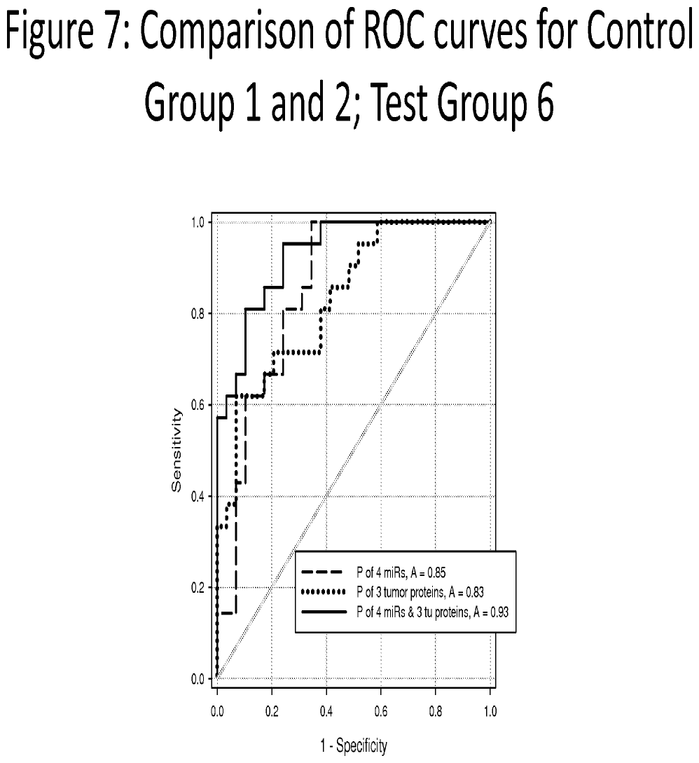

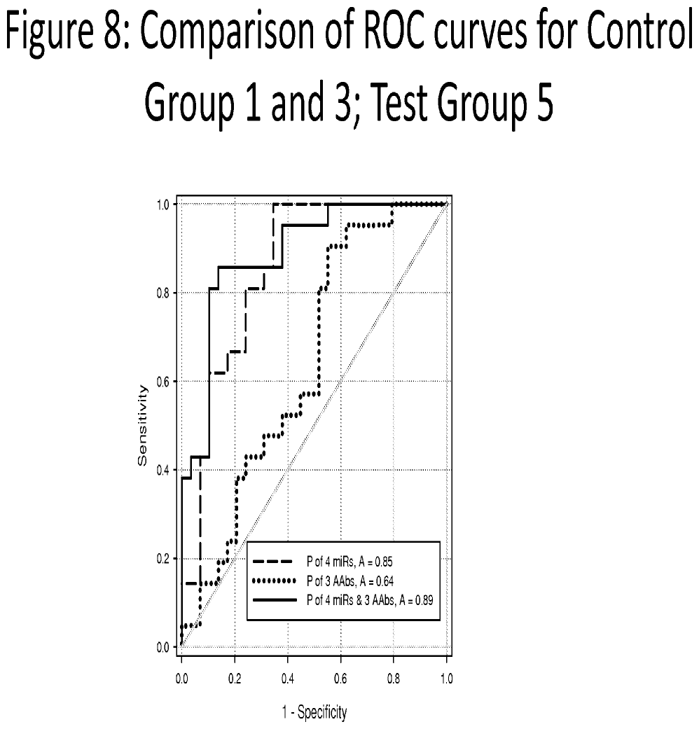

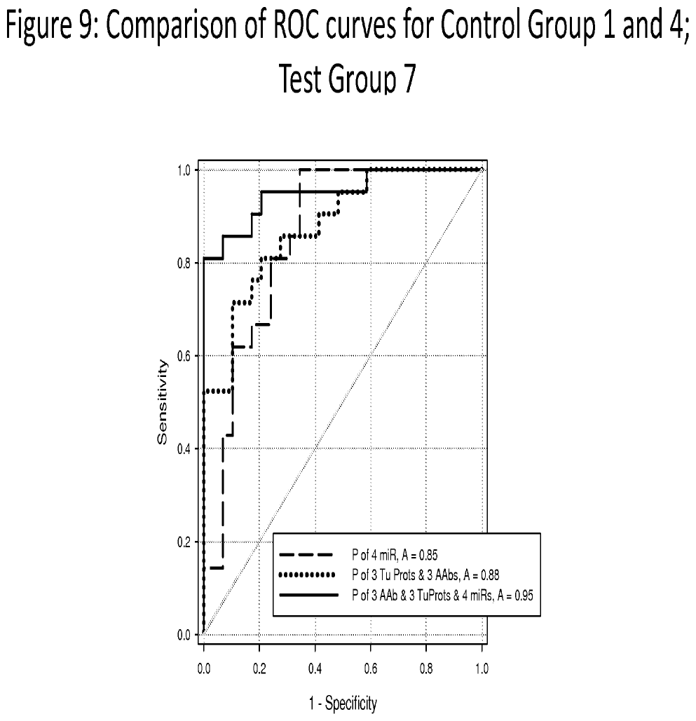

The data from this analysis clearly demonstrate that combining miRNA biomarkers with TP lung cancer biomarkers and/or AAB lung cancer biomarkers increased the sensitivity for lung cancer that was not expected from the sensitivity of the Control Groups. In this instance, Group 5 showed an increase in sensitivity over Control Groups 1 and 3 of 19% and 62% respectively. The comparison of ROC curves for Control Groups 1, 3 and Test Group 5 are shown in FIG. 8; the AUC value for Test Group 5 was 0.89. Group 6 showed an increase in sensitivity over Control Groups 1 and 2 of 19% for both groups. The comparison of ROC curves for Control Groups 1, 2 and Test Group 5 are shown in FIG. 7; the AUC value for Test Group 5 was 0.93. Group 7 showed an increase in sensitivity over Control Groups 1 and 4 of 24% and 14% respectively. The comparison of ROC curves for Control Groups 1, 4 and Test Group 7 are shown in FIG. 9; the AUC value for Test Group 7 was 0.95.

In certain embodiments, the inclusion of miRNA lung cancer biomarkers in a panel with TP and/or AAB lung cancer biomarkers increased the sensitivity for lung cancer by at least 3%, by at least 5%, by at least 10%, by at least 15%, by at least 20%, by at least 30%, by at least 40%, by at least 50% or by at least 60% as compared to miRNA lung cancer biomarkers alone (Control Group 1), or as compared to TP lung cancer biomarkers alone (Control Group 2), or as compared to AAB lung cancer biomarkers alone (Control Group 3), or as compared to a combination of AAB and TP lung cancer biomarkers in a panel (Control Group 4). Provided herein is a method for improving the sensitivity and/or diagnostic accuracy for lung cancer.

In certain embodiments, a panel of lung cancer biomarkers including at least one miRNA lung cancer biomarker and an additional TP and/or AAB lung cancer biomarker provides at least 80% sensitivity (at 80% specificity), at least 85% sensitivity, at least 90% sensitivity, or at least 95% sensitivity for lung cancer. In another embodiment, a panel of lung cancer biomarkers including at least one miRNA lung cancer biomarker and an additional TP and/or AAB lung cancer biomarker provides an AUC value of at least 0.89 for lung cancer.

In certain embodiments, the inclusion of miRNA lung cancer biomarkers in a panel with TP and/or AAB lung cancer biomarkers, when measured as a panel, are used to predict whether or not a patient is positive for lung cancer. In this instance, the lung cancer biomarkers (at least one miRNA biomarker and at least one of TP and/or AAB lung cancer biomarkers) are measured and a probability value calculated for cancer from the measured lung cancer biomarker levels. That value is then compared to a set threshold value to determine whether or not the probability value is above or below the threshold value. In alternative embodiments, the probability value is a percentage number that is not compared threshold but used as an absolute value (e.g. 60% chance a patient is positive for lung cancer). When compared to a threshold a prediction as to positive or negative for lung cancer can be made by concluding, if the probability value is above the threshold value, that the patient is positive for lung cancer, or concluding, if the probability value is below the threshold value, that the patient is negative for lung cancer.

In further embodiments, any of the methods of the present disclosure a threshold may be set, based on for example sensitivity value, AUC value, or probability value, wherein a measured panel in a sample below the set threshold is negative for lung cancer and a measured value above the set threshold is positive for lung cancer. In that way, the present methods and panels can diagnose lung cancer based on an acceptable sensitivity (e.g. greater than 65%) or a combination of sensitivity and specificity represented as an AUC value (e.g. set threshold of 0.80 for an AUC value).

The disclosure herein provides methods and compositions, including panels of biomarkers, for lung cancer screening including; diagnosing lung cancer in a patient and/or determining the likelihood of cancer in a patient and/or categorizing a patient's risk for lung cancer and/or determining a patient's increased risk for lung cancer and/or predicting whether a patient is positive for lung cancer or not. See Table 2 below in Example 2. As used herein, the term “increased risk” refers to an increase for the presence of the cancer as compared to the known prevalence of that particular cancer across a population cohort.

In one aspect the patient is asymptomatic with respect to lung cancer. In another aspect the patient has one or more risk factors (e.g. age, history of smoking, etc.).

### B) Definitions

As used herein, the terms “a” or “an” are used, as is common in patent documents, to include one or more than one, independent of any other instances or usages of “at least one” or “one or more.”

As used herein, the term “or” is used to refer to a nonexclusive or, such that “A or B” includes “A but not B,” “B but not A,” and “A and B,” unless otherwise indicated.

As used herein, the term “about” is used to refer to an amount that is approximately, nearly, almost, or in the vicinity of being equal to or is equal to a stated amount, e.g., the state amount plus/minus about 5%, about 4%, about 3%, about 2% or about 1%.

As used herein, the term “asymptomatic” refers to a patient or human subject that has not previously been diagnosed with the same cancer that their risk of having is now being quantified and categorized. For example, human subjects may shows signs such as coughing, fatigue, pain, etc., but had not been previously diagnosed with lung cancer but are now undergoing screening to categorize their increased risk for the presence of cancer and for the present methods are still considered “asymptomatic”.

As used herein, the term “AUC” refers to the Area Under the Curve, for example, of a ROC Curve. That value can assess the merit of a test on a given sample population with a value of 1 representing a good test ranging down to 0.5 which means the test is providing a random response in classifying test subjects. Since the range of the AUC is only 0.5 to 1.0, a small change in AUC has greater significance than a similar change in a metric that ranges for 0 to 1 or 0 to 100%. When the % change in the AUC is given, it will be calculated based on the fact that the full range of the metric is 0.5 to 1.0. A variety of statistics packages can calculate AUC for an ROC curve, such as, SigmaPlot 12.5, JMP™ or Analyse-It™. AUC can be used to compare the accuracy of the classification algorithm across the complete data range. Classification algorithms with greater AUC have, by definition, a greater capacity to classify unknowns correctly between the two groups of interest (disease and no disease). The classification algorithm maybe as simple as the measure of a single molecule or as complex as the measure and integration of multiple molecules.

As used herein, the terms “biological sample” and “test sample” refer to all biological fluids and excretions isolated from any given subject. In the context of the present invention such samples include, but are not limited to, blood, blood serum, blood plasma, urine, tears, saliva, sweat, biopsy, ascites, cerebrospinal fluid, milk, lymph, bronchial and other lavage samples, or tissue extract samples. In certain embodiments, blood, serum, plasma and bronchial lavage or other liquid samples are convenient test samples for use in the context of the present methods.

As used herein, the terms “cancer” and “cancerous” refer to or describe the physiological condition in mammals that is typically characterized by unregulated cell growth. Examples of cancer include but are not limited to, lung cancer, breast cancer, colon cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, and brain cancer.

As used herein, the term “cancer risk factors” refers to biological or environmental influences that are known risks associated with a particular cancer. These cancer risk factors include, but are not limited to, a family history of cancer (e.g. breast cancer), age, weight, sex, history of smoking tobacco, exposure to asbestos, exposure to radiation, etc. In certain embodiments, cancer risk factors for lung cancer are a human subject aged 50 years or older with a history of smoking tobacco.

As used herein, the term “cohort” refers to a group or segment of human subjects with shared factors or influences, such as age, family history, cancer risk factors, environmental influences, etc. In one instance, as used herein, a “cohort” refers to a group of human subjects with shared cancer risk factors; this is also referred to herein as a “disease cohort”. In another instance, as used herein, a “cohort” refers to a normal population group matched, for example by age, to the cancer risk cohort; also referred to herein as a “normal cohort”.

As used herein, the terms “differentially expressed gene,” “differential gene expression” and their synonyms, which are used interchangeably, are used in the broadest sense and refers to a gene and/or resulting protein whose expression is activated to a higher or lower level in a subject suffering from a disease, specifically cancer, such as lung cancer, relative to its expression in a normal or control subject. The terms also include genes whose expression is activated to a higher or lower level at different stages of the same disease. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example. Differential gene expression may include a comparison of expression between two or more genes or their gene products (e,g, proteins), or a comparison of the ratios of the expression between two or more genes or their gene products, or even a comparison of two differently processed products of the same gene, which differ between normal subjects and subjects suffering from a disease, specifically cancer, or between various stages of the same disease. Differential expression includes both quantitative, as well as qualitative, differences in the temporal or cellular expression pattern in a gene or its expression products among, for example, normal and diseased cells, or among cells which have undergone different disease events or disease stages.

As used herein, the term “gene expression profiling” is used in the broadest sense, and includes methods of quantification of mRNA and/or protein levels in a biological sample.

As used herein, the term “increased risk” refers to an increase in the risk level, for a human subject after testing, for the presence of a cancer relative to a population's known prevalence of a particular cancer before testing. In other words, a human subject's risk for cancer before testing may be 2% (based on the understood prevalence of cancer in the population), but after testing (based on the measure of biomarkers) their risk for the presence of cancer may be 30% or alternatively reported as an increase of 15 times compared to the cohort.

As used herein, the term “decreased risk” refers to a decrease in the risk level, for a human subject after testing, for the presence of a cancer relative to a population's known prevalence of a particular cancer before testing. In this instance, “decreased risk” refers to a change in risk level relative to a population before testing.

As used herein, the term “lung cancer” refers to a cancer state associated with the pulmonary system of any given subject. In the context of the present invention, lung cancers include, but are not limited to, adenocarcinoma, epidermoid carcinoma, squamous cell carcinoma, large cell carcinoma, small cell carcinoma, non-small cell carcinoma, and bronchoalveolar carcinoma. Within the context of the present invention, lung cancers may be at different stages, as well as varying degrees of grading. Methods for determining the stage of a lung cancer or its degree of grading are well known to those skilled in the art.

As used herein, the terms “marker”, “biomarker” (or fragment thereof) and their synonyms, which are used interchangeably, refer to molecules that can be evaluated in a sample and are associated with a physical condition. For example, a marker includes expressed genes or their products (e.g. proteins) or autoantibodies to those proteins that can be detected from a human samples, such as blood, serum, solid tissue, and the like, that, that is associated with a physical or disease condition or microRNA, or any combination thereof. Such biomarkers include, but are not limited to, biomolecules comprising nucleotides, amino acids, sugars, fatty acids, steroids, metabolites, polypeptides, proteins (such as, but not limited to, antigens and antibodies), carbohydrates, lipids, hormones, antibodies, regions of interest which serve as surrogates for biological molecules, combinations thereof (e.g., glycoproteins, ribonucleoproteins, lipoproteins) and any complexes involving any such biomolecules, such as, but not limited to, a complex formed between an antigen and an autoantibody that binds to an available epitope on said antigen. The term “biomarker” can also refer to a portion of a polypeptide (parent) sequence that comprises at least 5 consecutive amino acid residues, preferably at least 10 consecutive amino acid residues, more preferably at least 15 consecutive amino acid residues, and retains a biological activity and/or some functional characteristics of the parent polypeptide, e.g. antigenicity or structural domain characteristics. The present markers refer to both tumor antigens present on or in cancerous cells or those that have been shed from the cancerous cells into bodily fluids such as blood or serum. The present markers, as used herein, also refer to autoantibodies produced by the body to those tumor antigens and circulating miRNA. In one aspect, a “marker” as used herein refers to miRNA and tumor proteins (TP) and/or autoantibodies (AAB) that are capable of being detected in serum of a human subject. It is also understood in the present methods that use of the markers in a panel may each contribute equally to the composite score or certain biomarkers may be weighted wherein the markers in a panel contribute a different weight or amount to the final composite score.

It is understood that some tumor protein (TP) type biomarkers for lung cancer may come from non-tumor cells that interact with tumor cells. In that instance, the immune system can produce, not only autoantibodies, but a wide spectrum of cell signaling molecules (e.g., cytokines etc.). The origin of circulating protein biomarkers identified in most studies cannot be proved, although their overexpression in cancer cells may be associated with elevated blood levels. The term “tumor protein” or TP may be used herein interchangeably with “tumor associated protein” or “lung cancer associated proteins” (LCAP).

As used herein, the term “normalization” and its derivatives, when used in conjunction with measurement of biomarkers across samples and time, refer to mathematical methods where the intention is that these normalized values allow the comparison of corresponding normalized values from different datasets in a way that eliminates or minimizes differences and gross influences.

As used herein, the terms “panel of markers”, “panel of biomarkers” and their synonyms, which are used interchangeably, refer to more than one marker that can be detected from a human sample that together, are associated with the presence of a particular cancer. It is understood that a panel refers to the measurement of at least one miRNA biomarker and at least one additional AAB and/or TP lung cancer biomarker from the same sample, but that they do not necessarily need to be measured at the same time, or from the same aliquot of sample.

As used herein, the term “microRNA” (miRNA or miR) includes human miRNAs, mature single stranded miRNAs, precursor miRNAs (pre-miR), and variants thereof, which may be naturally occurring. In some instances, the term “miRNA” also includes primary miRNA transcripts and duplex miRNAs. Unless otherwise noted, when used herein, the name of a specific miRNA refers to the mature miRNA of a precursor miRNA. For example, miR-122a refers to a mature miRNA sequence derived from pre-miR-122. The sequences for particular miRNAs, including human mature and precursor sequences, are reported in the miRBase::Sequences Database (http://microrna.sanger.ac.uk (version 15 released April 2010); Griffiths-Jones et al., Nucleic Acids Research, 2008, 36, Database Issue, D154-D158; Griffiths-Jones et al., Nucleic Acids Research, 2006, 34, Database Issue, D140-D144; and Griffiths-Jones, Nucleic Acids Research, 2004, 32, Database Issue, D109-D111). For certain miRNAs, a single precursor contains more than one mature miRNA sequence. In other instances, multiple precursor miRNAs contain the same mature sequence. In some instances, mature miRNAs have been re-named based on new scientific consensus. For example, miR-213, as used herein, refers to a mature miRNA from pre-miR-181a-1, and is also called miR-181a*. Other miRNAs that have been re-named include miR-189 (also called miR-24*), which comes from pre-miR-24-1; miR-368 (also called miR-376c); and miR-422b (also called miR-378*). The skilled artisan will appreciate that scientific consensus regarding the precise nucleic acid sequence for a given miRNA, in particular for mature forms of the miRNAs, may change with time. MiRNAs detected by assays of this application include naturally occurring sequences for the miRNAs.

As used herein, the term “pathology” of (tumor) cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

As used herein, the term “known prevalence of cancer” refers to a prevalence of a cancer in a population before the human subject is tested using the present methods. This known prevalence of cancer, can be a prevalence reported in the literature based on retrospective data or an algorithm applied to that prevalence where in the algorithm takes into account factors such as age and more immediate and relevant history. In this instance, a known prevalence of cancer in a cohort refers to a risk of having cancer prior to being tested by the present methods.

As used herein, the term “a positive predictive score,” “a positive predictive value,” or “PPV” refers to the likelihood that a score within a certain range on a biomarker test is a true positive result. This is also referred to herein as a probability of cancer, represented as a percentage. It is defined as the number of true positive results divided by the number of total positive results. True positive results can be calculated by multiplying the test Sensitivity times the Prevalence of disease in the test population. False positives can be calculated by multiplying (1 minus the Specificity) times (1−the prevalence of disease in the test population). Total positive results equal True Positives plus False Positives.

As used herein, the term “probability of cancer”, refers to a probability or likelihood (e.g. represented as a percentage) that a patient, after screening using the present methods, is positive for the presence of lung cancer.

As used herein the term, “Receiver Operating Characteristic Curve,” or, “ROC curve,” is a plot of the performance of a particular feature for distinguishing two populations, patients with lung cancer, and controls, e.g., those without lung cancer. Data across the entire population (namely, the patients and controls) are sorted in ascending order based on the value of a single feature. Then, for each value for that feature, the true positive and false positive rates for the data are determined. The true positive rate is determined by counting the number of cases above the value for that feature under consideration and then dividing by the total number of patients. The false positive rate is determined by counting the number of controls above the value for that feature under consideration and then dividing by the total number of controls.

ROC curves can be generated for a single feature as well as for other single outputs, for example, a combination of two or more features that are combined (such as, added, subtracted, multiplied etc.) to provide a single combined value which can be plotted in a ROC curve.

The ROC curve is a plot of the true positive rate (sensitivity) of a test against the false positive rate (1−specificity) of the test. ROC curves provide another means to quickly screen a data set.

As used herein, the term “screening” refers to a strategy used in a population to identify an unrecognized cancer in asymptomatic subjects, for example those without signs or symptoms of the cancer. As used herein, a cohort of the population (e.g. smokers aged 50 or older) are screened for a particular cancer (e.g. lung cancer) wherein the present methods are applied to determine the likelihood and/or risk to those asymptomatic subjects for the presence of the cancer.

As used herein, the term “sensitivity” refers to statistical analysis that measures the proportion of positives which are correctly identified as positives: true positives. The higher the sensitivity the fewer false negatives are identified. The sensitivity, at a designated specificity cutoff (e.g., 80%), of a biomarker or panels or biomarkers for a particular disease (e.g., lung cancer) can be measured and used to assess a patient's risk for the particular disease.

As used herein, the term “specificity” refers to statistical analysis that measures the proportion of negatives which are correctly identified as negative; true negatives. The higher the specificity the lower the false positive rate. The higher the combined specificity (e.g., 80%) and sensitivity (e.g., at least 80%) the better predictor a biomarker, or panel of biomarkers, are for correctly identifying lung cancer with clinical utility.

As used herein, the term “subject” refers to an animal, preferably a mammal, including a human or non-human. The terms “patient” and “human subject” may be used interchangeably herein.

As used herein, the term “tumor,” refers to all neoplastic cell growth and proliferation, whether malignant or benign, and all pre-cancerous and cancerous cells and tissues.

As used herein, the phrase “Weighted Scoring Method” refers to a method that involves converting the measurement of one biomarker that is identified and quantified in a test sample into one of many potential scores. A ROC curve can be used to standardize the scoring between different markers by enabling the use of a weighted score based on the inverse of the false positive % defined from the ROC curve. The weighted score can be calculated by multiplying the AUC by a factor for a marker and then dividing by the false positive % based on a ROC curve. The weighted score can be calculated using the formula:

Weighted Score=(AUCₓ×factor)/(1−% specificityₓ)

wherein x is the marker; the, “factor,” is a real number (such as 0, 1, 2, 3, 4, 5, 6, 7, 8, 9, 10, 11, 12, 13, 14, 15, 16, 17, 18, 19, 20, 21, 22, 23, 24, 25 and so on) throughout the panel; and the, “specificity,” is a chosen value that does not exceed 95% (e.g., 80%). Multiplication of a factor for the panel allows the user to scale the weighted score. Hence, the measurement of one marker can be converted into as many or as few scores as desired.

The weighting provides higher scores for biomarkers with a low false positive rate (thereby having higher specificity) for the population of interest. The weighting paradigm can comprise electing levels of false positivity (1−specificity) below which the test will result in an increased score. Thus, markers with high specificity can be given a greater score or a greater range of scores than markers that are less specific.

Foundation for assessing the parameters for weighing can be obtained by determining presence of a marker in a population of patients with lung cancer and in normal individuals. The information (data) obtained from all the samples are used to generate a ROC curve and to create an AUC for each biomarker. A number of predetermined cutoffs and a weighted score are assigned to each biomarker based on the % specificity. That calculus provides a stratification of aggregate scores, and those scores can be used to define ranges that correlate to arbitrary risk categories of whether one has a higher or lower risk of having lung cancer. The number of categories can be a design choice or may be driven by the data.

C) Biomarkers

The present disclosure is directed to a panel of miRNA lung cancer biomarkers comprising at least one additional lung cancer biomarker selected from the group of tumor protein (TP) or autoantibody (AAB) lung cancer biomarkers and their use in screening for lung cancer. As used herein “screening for lung cancer” refers to diagnosing lung cancer in a patient and/or determining the likelihood of cancer in a patient and/or categorizing a patient's risk for lung cancer and/or determining a patient's increased risk for lung cancer.

In certain embodiments are provided lung cancer biomarker panels, wherein the panel comprises at least one miRNA lung cancer biomarker and at least one TP lung cancer biomarker and at least one AAB lung cancer biomarker. In certain other embodiments are provided lung cancer biomarker panels, wherein the panel comprises at least one miRNA lung cancer biomarker and at least one TP lung cancer biomarker. In certain other embodiments are provided lung cancer biomarker panels, wherein the panel comprises at least one miRNA lung cancer biomarker and at least one AAB lung cancer biomarker.

In certain embodiments, the panels comprise at least one, at least two, at least three, at least four, at least five, at least six, at least seven, at least eight, at least nine, at least 10, at least 15, at least 20, at least 30, at least 40 or at least 50 miRNA lung cancer biomarkers. In one aspect the panel further comprises at least one, at least two, at least three, at least four, at least five, at least six, at least seven, at least eight, at least nine, at least ten (10), at least 15, at least 20, at least 30, at least 40 or at least 50 TP lung cancer biomarkers. In another aspect, the panel further comprises at least one, at least two, at least three, at least four, at least five, at least six, at least seven, at least eight, at least nine, at least 10, at least 15, at least 20, at least 30, at least 40 or at least 50 AAB lung cancer biomarkers.

Both the total number of biomarkers in the panel as well as the total number from each group (miRNA, TP and AAB) may be optimized as feasible to obtain clinical relevancy wherein the panel has increased sensitivity as compared to a panel with only one group (miRNA, TP or AAB) of lung cancer biomarkers (e.g. greater than 80% sensitivity at 80% specificity). In this instance, a panel may comprise X number of miRNA lung cancer biomarkers and Y number of TP and/or AAB lung cancer biomarkers, wherein X and Y may be the same or different and are at least one to at least about 50 lung cancer biomarkers.

In certain embodiments the lung cancer panel comprises X miRNA lung cancer biomarkers and Y TP lung cancer biomarkers. In another embodiment, the lung cancer biomarker panel comprises X miRNA lung cancer biomarkers and Y′ AAB lung cancer biomarkers. In yet another embodiment, the lung cancer biomarker panel comprises X miRNA lung cancer biomarkers, Y TP lung cancer biomarkers and Y′ AAB lung cancer biomarkers. X, Y and Y′ represent at least one to about at least 50 lung cancer biomarkers and may be the same or different in each panel.

In one embodiment, the panel comprises four miRNA lung cancer biomarkers and three TP lung cancer biomarkers. In another embodiment, the panel comprises four miRNA lung cancer biomarkers and three AAB lung cancer biomarkers. In yet another embodiment, the panel comprises four miRNA lung cancer biomarkers and three TP lung cancer biomarkers and three AAB lung cancer biomarkers.

In certain embodiments, the panel comprises about 1 to about 10 miRNA lung cancer biomarkers, about 1 to about 10 TP lung cancer biomarkers and/or about 1 to about 10 AAB lung cancer biomarkers. In one aspect the panel comprises one miRNA lung cancer biomarker, two miRNA lung cancer biomarkers, three miRNA lung cancer biomarkers, four miRNA lung cancer biomarkers, five miRNA lung cancer biomarkers, six miRNA lung cancer biomarkers, seven miRNA lung cancer biomarkers, eight miRNA lung cancer biomarkers, nine miRNA lung cancer biomarkers or ten (10) miRNA lung cancer biomarkers in combination with about 1 to about 10 TP lung cancer biomarkers and/or about 1 to about 10 AAB lung cancer biomarkers.

In another aspect, the panel comprises one miRNA lung cancer biomarker, two miRNA lung cancer biomarkers, three miRNA lung cancer biomarkers, four miRNA lung cancer biomarkers, five miRNA lung cancer biomarkers, six miRNA lung cancer biomarkers, seven miRNA lung cancer biomarkers, eight miRNA lung cancer biomarkers, nine miRNA lung cancer biomarkers or ten (10) miRNA lung cancer biomarkers in combination with one TP lung cancer biomarker, two TP lung cancer biomarker, three TP lung cancer biomarker, four, TP lung cancer biomarker, five TP lung cancer biomarker, six TP lung cancer biomarker, seven TP lung cancer biomarkers, eight TP lung cancer biomarkers, nine TP lung cancer biomarkers or (10) TP lung cancer biomarkers and/or about 1 to about 10 AAB lung cancer biomarkers.

In yet another aspect, the panel comprises one miRNA lung cancer biomarker, two miRNA lung cancer biomarkers, three miRNA lung cancer biomarkers, four miRNA lung cancer biomarkers, five miRNA lung cancer biomarkers, six miRNA lung cancer biomarkers, seven miRNA lung cancer biomarkers, eight miRNA lung cancer biomarkers, nine miRNA lung cancer biomarkers or 10 miRNA lung cancer biomarkers in combination with one TP lung cancer biomarker, two TP lung cancer biomarkers, three TP lung cancer biomarkers, four TP lung cancer biomarkers, five TP lung cancer biomarkers, six TP lung cancer biomarkers, seven TP lung cancer biomarkers, eight TP lung cancer biomarkers, nine TP lung cancer biomarkers or ten (10) TP lung cancer biomarkers and/or one AAB lung cancer biomarker, two AAB lung cancer biomarkers, three AAB lung cancer biomarkers, four AAB lung cancer biomarkers, five AAB lung cancer biomarkers, six AAB lung cancer biomarkers, seven AAB lung cancer biomarkers, eight AAB lung cancer biomarkers, nine AAB lung cancer biomarkers or 10 AAB lung cancer biomarkers.

It is understood that for any of the lung cancer panels described herein, the panel measures the biomarker listed in the panel and that the panel does not comprise that biomarker but rather the means to measure the level in a sample of that stated biomarker.

However, before measurement can be performed a panel of biomarkers needs to be selected for screening lung cancer. Many biomarkers are known for lung cancer and a panel can be selected, or as was done by the present Applicants, a panel can be selected based on measurement of individual markers in retrospective clinical samples wherein a panel is generated based on empirical data for lung cancer.

Examples of biomarkers that can be employed include measurable molecules, for example, in a body fluid sample, such as, antibodies, antigens, small molecules, proteins, hormones, genes and so on, wherein the present lung cancer panel comprises at least one miRNA lung cancer biomarker and at least one additional lung cancer biomarker from the TP and/or AAB group of lung cancer biomarkers.

MicroRNA Lung Cancer Biomarkers

Micro-RNAs (miRNA or miR) that are proposed to be circulating markers for lung cancer include miR-21, miR-126, miR-210, miR-486-5p (Shen, J., et al., Plasma microRNAs as potential biomarkers for non-small-cell lung cancer. Lab Invest, 2011. 91(4): p. 579-587); miR-15a, miR-15b, miR-27b, miR-142-3p, miR-301 (Hennessey, P. T., et al., Serum microRNA Biomarkers for Detection of Non-Small Cell Lung Cancer. PLoS ONE, 2012. 7(2): p. e32307); let-7b, let-7c, let-7d, let-7e, miR-10a, miR-10b, miR-130b, miR-132, miR-133b, miR-139, miR-143, miR-152, miR-155, miR-15b, miR-17-5p, miR-193, miR-194, miR-195, miR-196b, miR-199a*, miR-19b, miR-202, miR-204, miR-205, miR-206, miR-20b, miR-21, miR-210, miR-214, miR-221, miR-27a, miR-27b, miR-296, miR-29a, miR-301, miR-324-3p, miR-324-5p, miR-339, miR-346, miR-365, miR-378, miR-422a, miR-432, miR-485-3p, miR-496, miR-497, miR-505, miR-518b, miR-525, miR-566, miR-605, miR-638, miR-660, and miR-93 [US Patent Publ. No. 2011/0053158]; hsa-miR-361-5p, hsa-miR-23b, hsa-miR-126, hsa-miR-527, hsa-miR-29a, hsa-let-7i, hsa-miR-19a, hsa-miR-28-5p, hsa-miR-185*, hsa-miR-23a, hsa-miR-1914*, hsa-miR-29c, hsa-miR-505*, hsa-let-7d, hsa-miR-378, hsa-miR-29b, hsa-miR-604, hsa-miR-29b, hsa-let-7b, hsa-miR-299-3p, hsa-miR-423-3p, hsa-miR-18a*, hsa-miR-1909, hsa-let-7c, hsa-miR-15a, hsa-miR-425, hsa-miR-93*, hsa-miR-665, hsa-miR-30e, hsa-miR-339-3p, hsa-miR-1307, hsa-miR-625*, hsa-miR-193a-5p, hsa-miR-130b, hsa-miR-17*, hsa-miR-574-5p and hsa-miR-324-3p. (US Patent Publ. No. 2012/0108462); miR-20a, miR-24, miR-25, miR-145, miR-152, miR-199a-5p, miR-221, miR-222, miR-223, miR-320 (Chen, X., et al., Identification of ten serum microRNAs from a genome-wide serum microRNA expression profile as novel noninvasive biomarkers for non-small cell lung cancer diagnosis. International Journal of Cancer, 2012. 130(7): p. 1620-1628); hsa-let-7a, hsa-let-7b, hsa-let-7d, hsa-miR-103, hsa-miR-126, hsa-miR-133b, hsa-miR-139-5p, hsa-miR-140-5p, hsa-miR-142-3p, hsa-miR-142-5p, hsa-miR-148a, hsa-miR-148b, hsa-miR-17, hsa-miR-191, hsa-miR-22, hsa-miR-223, hsa-miR-26a, hsa-miR-26b, hsa-miR-28-5p, hsa-miR-29a, hsa-miR-30b, hsa-miR-30c, hsa-miR-32, hsa-miR-328, hsa-miR-331-3p, hsa-miR-342-3p, hsa-miR-374a, hsa-miR-376a, hsa-miR-432-staR, hsa-miR-484, hsa-miR-486-5p, hsa-miR-566, hsa-miR-92a, hsa-miR-98 (Bianchi, F., et al., A serum circulating miRNA diagnostic test to identify asymptomatic high-risk individuals with early stage lung cancer. EMBO Molecular Medicine, 2011. 3(8): p. 495-503); miR-190b, miR-630, miR-942, and miR-1284 (Patnaik, S. K., et al., MicroRNA Expression Profiles of Whole Blood in Lung Adenocarcinoma. PLoS ONE, 2012. 7(9): p. e46045).

In a particular embodiment, the lung cancer biomarker panel comprises at least one of miR-21, miR-126, miR-210, miR-486.

Tumor Protein (TP) and Autoantibody (AAB) Lung Cancer Biomarkers

A research effort to identify panels of biomarkers that included a survey of known tumor protein biomarkers coupled with a discovery project for novel lung cancer specific biomarkers was previously conducted (PCT Publ. No. WO 2009/006323, incorporated herein by reference). This work indicates that a combination of markers can be used to increase sensitivity of testing for lung cancer without greatly affecting the specificity of the test. To accomplish this, biomarkers were tested and analyzed culminating in the establishment of a panel of six biomarkers (three TP and three AAB) that in the aggregate yield significant sensitivity and specificity for the early detection of lung cancer. This panel demonstrated a 76.2% sensitivity at 80% specificity for lung cancer when used on the Samples of Example 1. See FIG. 4. As disclosed herein, Applicants provide an improvement by combining miRNA biomarkers with TP and/or AAB lung cancer biomarkers for screening patients for lung cancer. The inclusion of miRNA biomarkers in this panel provides a sensitivity (at 80% specificity) of 86% and 91%, an improvement compared to the TP and AAB panel as well as the miRNA panel.

In one embodiment, the panel of markers is selected from anti-p53, anti-NY-ESO-1, anti-ras, anti-Neu, anti-MAPKAPK3, cytokeratin 8, cytokeratin 19, cytokeratin 18, CEA, CA125, CA15-3, CA19-9, Cyfra 21-1, serum amyloid A, proGRP and ai-anti-trypsin (US Patent Publ. Nos. 2012/0071334; 2008/0160546; 2008/0133141; 2007/0178504 (each herein incorporated by reference)). Many circulating proteins have more recently been identified as possible biomarkers for the occurrence of lung cancer, for example the proteins CEA, RBP4, hAAT, SCCA [Patz, E. F., et al., Panel of Serum Biomarkers for the Diagnosis of Lung Cancer. Journal of Clinical Oncology, 2007. 25(35): p. 5578-5583.]; the proteins IL6, IL-8 and CRP [Pine, S. R., et al., Increased Levels of Circulating Interleukin 6, Interleukin 8, C-Reactive Protein, and Risk of Lung Cancer. Journal of the National Cancer Institute, 2011. 103(14): p. 1112-1122.]; the proteins TNF-α, CYFRA 21-1, IL-1ra, MMP-2, monocyte chemotactic protein-1 & sE-selectin [Farlow, E. C., et al., Development of a Multiplexed Tumor-Associated Autoantibody-Based Blood Test for the Detection of Non-Small Cell Lung Cancer. Clinical Cancer Research, 2010. 16(13): p. 3452-3462.]; the proteins prolactin, transthyretin, thrombospondin-1, E-selectin, C-C motif chemokine 5, macrophage migration inhibitory factor, plasminogen activator inhibitor, receptor tyrosine-protein kinase, erbb-2, cytokeratin fragment 21.1, and serum amyloid A [Bigbee, W. L. P., et al., —A Multiplexed Serum Biomarker Immunoassay Panel Discriminates Clinical Lung Cancer Patients from High-Risk Individuals Found to be Cancer-Free by CT Screening [Journal of Thoracic Oncology April, 2012. 7(4): p. 698-708.]; the proteins EGF, sCD40 ligand, IL-8, MMP-8 [Izbicka, E., et al., Plasma Biomarkers Distinguish Non-small Cell Lung Cancer from Asthma and Differ in Men and Women. Cancer Genomics—Proteomics, 2012. 9(1): p. 27-35.].

Novel ligands that bind to circulating, lung-cancer associated proteins which are possible biomarkers include nucleic acid aptamers to bind cadherin-1, CD30 ligand, endostatin, HSP90a, LRIG3, MIP-4, pleiotrophin, PRKCI, RGM-C, SCF-sR, sL-selectin, and YES [Ostroff, R. M., et al., Unlocking Biomarker Discovery: Large Scale Application of Aptamer Proteomic Technology for Early Detection of Lung Cancer. PLoS ONE, 2010. 5(12): p. e150031 and monoclonal antibodies that bind leucine-rich alpho-2 glycoprotein 1 (LRG1), alpha-1 antichymotrypsin (ACT), complement C9, haptoglobin beta chain [Guergova-Kuras, M., et al., Discovery of Lung Cancer Biomarkers by Profiling the Plasma Proteome with Monoclonal Antibody Libraries. Molecular & Cellular Proteomics, 2011. 10(12).]; and the protein [Higgins, G., et al., Variant Cizl is a circulating biomarker for early-stage lung cancer. Proceedings of the National Academy of Sciences, 2012.].

Autoantibodies that are proposed to be circulating markers for lung cancer include p53, NY-ESO-1, CAGE, GBU4-5, Annexin 1, and SOX2 [Lam, S., et al., EarlyCDT-Lung: An Immunobiomarker Test as an Aid to Early Detection of Lung Cancer. Cancer Prevention Research, 2011. 4(7): p. 1126-1134.] and IMPDH, phosphoglycerate mutase, ubiquillin, Annexin I, Annexin II, and heat shock protein 70-9B (HSP70-9B) [Farlow, E. C., et al., Development of a Multiplexed Tumor-Associated Autoantibody-Based Blood Test for the Detection of Non-Small Cell Lung Cancer. Clinical Cancer Research, 2010. 16(13): p. 3452-3462.].

In a particular embodiment, the TP lung cancer biomarkers are selected from CEA, CA125, CA15-3, CA19-9, Cyfra 21-1, serum amyloid A, and proGRP. In another embodiment, the AAB lung cancer biomarkers are selected from anti-p53, anti-NY-ESO-1, anti-CAGE, anti-GBU4-5, anti-Annexin 1, anti-SOX2, anti-ras, anti-Neu, and anti-MAPKAPK3. In one embodiment, the lung cancer panel comprises at least one of anti-p53, anti-NY-ESO-1, or anti-MAPKAPK3. In another embodiment, the panel comprises at least one of CEA, Cyfra 21-1, or CA125.

In one embodiment, a panel of markers for lung cancer is selected from CEA (GenBank Accession CAE75559), CA125 (UniProtKB/Swiss-Prot: Q8WXI7.2), Cyfra 21-1 (NCBI Reference Sequence: NP_008850.1), anti-NY-ESO-1 (antigen NCBI Reference Sequence: NP_001318.1), anti-p53 (antigen GenBank: BAC16799.1) and anti-MAPKAPK3 (antigen NCBI Reference Sequence: NP_001230855.1), the first three are tumor marker proteins and the last three are autoantibodies.

Methods for Screening for Lung Cancer Using the Lung Cancer Biomarker Panels

In certain embodiments provided herein are methods for screening a patient for lung cancer. Screening, includes, but is not limited to using the present lung cancer biomarker panels for diagnosing lung cancer in a patient and/or determining the likelihood of cancer in a patient and/or categorizing a patient's risk for lung cancer and/or determining a patient's increased risk for lung cancer. In one aspect, the risk level is increased as compared to the population. In another aspect, the risk level is decreased as compared to the population. The asymptomatic patients that, after testing, have a quantified increased risk for the presence of cancer relative to the population are those that a physician may select for follow-on testing.

Therefore, in certain embodiments, are methods for assessing the likelihood that a patient has lung cancer, comprising 1) measuring a level of at least one miRNA lung cancer biomarker in a sample from the human subject; 2) measuring a level of at least one cancer biomarker selected from a tumor protein (TP) lung cancer biomarker or an autoantibody (AAB) lung cancer biomarker in a sample from the human subject; and 3) calculating a probability of cancer from said biomarker measurements, whereby the likelihood that a patient has lung cancer is determined.

One or more steps of the method described herein can be performed manually or can be completely or partially automated (for example, one or more steps of the method can be performed by a computer program or algorithm. If the method were to be performed via computer program or algorithm, then the performance of the method would further necessitate the use of the appropriate hardware, such as input, memory, processing, display and output devices, etc.). Methods for automating one or more steps of the method would be well within the skill of those in the art.

measuring biomarkers in a sample

The first step in the present method is measuring a panel of biomarkers, following sample collection, from a human subject, such as an asymptomatic human subject. There are many methods known in the art for measuring gene expression (e.g. mRNA), the resulting gene products (e.g. polypeptides or proteins), or non-coding RNAs that regulate gene expression (miRNA) that can be used in the present methods. The sample typically includes blood and is processed so that miRNA, TP and AAB lung cancer biomarkers are measured from a blood sample. In certain embodiments, the sample is from a patient suspected of having lung cancer or at risk of developing lung cancer. In one aspect, the patient is asymptomatic for lung cancer. The volume of plasma or serum obtained and used for the assay may be varied depending upon clinical intent.

One of skill in the art will recognize that many methods exist for obtaining and preparing serum samples. Generally, blood is drawn into a collection tube using standard methods and allowed to clot. The serum is then separated from the cellular portion of the coagulated blood. In some methods, clotting activators such as silica particles are added to the blood collection tube. In other methods, the blood is not treated to facilitate clotting. Blood collection tubes are commercially available from many sources and in a variety of formats (e.g., Becton Dickenson Vacutainer® tubes—SST™, glass serum tubes, or plastic serum tubes).

Methods for measuring protein biomarkers (or gene expression) is described for example in, PCT International Pat. Pub. No. WO 2009/006323; US Pub. No. 2012/0071334; US Pat. Publ. No. 2008/0160546; US Pat. Publ. No. 2008/0133141; US Pat. Pub. No. 2007/0178504 (each herein incorporated by reference) and teach a multiplex lung cancer assay using beads as the solid phase and fluorescence or color as the reporter in an immunoassay format. Hence, the degree of fluorescence (e.g., mean fluorescence intensity (MFI)) or color can be provided in the form of a qualitative score as compared to an actual quantitative value of reporter presence and amount.

For example, the presence and quantification of one or more antigens or antibodies in a test sample can be determined using one or more immunoassays that are known in the art. Immunoassays typically comprise: (a) providing an antibody (or antigen) that specifically binds to the biomarker (namely, an antigen or an antibody); (b) contacting a test sample with the antibody or antigen; and (c) detecting the presence of a complex of the antibody bound to the antigen in the test sample or a complex of the antigen bound to the antibody in the test sample.

Well known immunological binding assays include, for example, an enzyme linked immunosorbent assay (ELISA), which is also known as a “sandwich assay”, an enzyme immunoassay (EIA), a radioimmunoassay (RIA), a fluoroimmunoassay (FIA), a chemiluminescent immunoassay (CLIA) a counting immunoassay (CIA), a filter media enzyme immunoassay (MEIA), a fluorescence-linked immunosorbent assay (FLISA), agglutination immunoassays and multiplex fluorescent immunoassays (such as the Luminex Lab MAP), immunohistochemistry, etc. For a review of the general immunoassays, see also, Methods in Cell Biology: Antibodies in Cell Biology, volume 37 (Asai, ed. 1993); Basic and Clinical Immunology (Daniel P. Stites; 1991).

The immunoassay can be used to determine a test amount of an antigen in a sample from a subject. First, a test amount of an antigen in a sample can be detected using the immunoassay methods described above. If an antigen is present in the sample, it will form an antibody-antigen complex with an antibody that specifically binds the antigen under suitable incubation conditions described above. The amount of an antibody-antigen complex can be determined by comparing the measured value to a standard or control. The AUC for the antigen can then be calculated using techniques known, such as, but not limited to, a ROC analysis.

In another embodiment, gene expression of markers (e.g. mRNA) is measured in a sample from a human subject. For example, gene expression profiling methods for use with paraffin-embedded tissue include quantitative reverse transcriptase polymerase chain reaction (qRT-PCR), however, other technology platforms, including mass spectroscopy and DNA microarrays can also be used. These methods include, but are not limited to, PCR, Microarrays, Serial Analysis of Gene Expression (SAGE), and Gene Expression Analysis by Massively Parallel Signature Sequencing (MPSS).

Any methodology that provides for the measurement of a marker or panel of markers from a human subject is contemplated for use with the present methods. In certain embodiments, the sample from human subject is a tissue section such as from a biopsy. In another embodiment, the sample from the human subject is a bodily fluid such as blood, serum, plasma or a part or fraction thereof. In other embodiments, the sample is a blood or serum and the markers are proteins measured there from. In yet another embodiment, the sample is a tissue section and the markers are mRNA expressed therein. Many other combinations of sample forms from the human subjects and the form of the markers are contemplated.

US Patent Publ. No. 2011/0053158 teaches amplifying and measuring miRNA from serum samples. In certain methods, the blood is collected by venipuncture and processed within three hours after drawing to minimize hemolysis and minimize the release of miRNAs from intact cells in the blood. In some methods, blood is kept on ice until use. The blood may be fractionated by centrifugation to remove cellular components. In some embodiments, centrifugation to prepare serum can be at a speed of at least 500, 1000, 2000, 3000, 4000, or 5000×G. In certain embodiments, the blood can be incubated for at least 10, 20, 30, 40, 50, 60, 90, 120, or 150 minutes to allow clotting. In other embodiments, the blood is incubated for at most 3 hours. When using plasma, the blood is not permitted to coagulate prior to separation of the cellular and acellular components. Serum or plasma can be frozen after separation from the cellular portion of blood until further assayed.

Before analysis, RNA may be extracted from serum or plasma and purified using methods known in the art. Many methods are known for isolating total RNA, or for specifically extracting small RNAs, including miRNAs. The RNA may be extracted using commercially-available kits (e.g., Perfect RNA Total RNA Isolation Kit, Five Prime-Three Prime, Inc.; mirVana™ kits, Ambion, Inc.). Alternatively, RNA extraction methods for the extraction of mammalian intracellular RNA or viral RNA may be adapted, either as published or with modification, for extraction of RNA from plasma and serum. RNA may be extracted from plasma or serum using silica particles, glass beads, or diatoms, as in the method or adaptations described in U.S. Patent Publ. No. 2008/0057502.

In certain embodiments, the level of the miRNA marker will be compared to a control to determine whether the level is reduced or elevated. The control may be an external control, such as a miRNA in a serum or plasma sample from a subject known to be free of lung disease. The external control may be a sample from a normal (non-diseased) subject or from a patient with benign lung disease. In other circumstances, the external control may be a miRNA from a non-serum sample like a tissue sample or a known amount of a synthetic RNA. The external control may be a pooled, average, or individual sample; it may be the same or different miRNA as one being measured. An internal control is a marker from the same serum or plasma sample being tested, such as a miRNA control. See, e.g., US Patent Publ. No. 2009/0075258, which is incorporated by reference in its entirety.

Many methods of measuring the levels or amounts of miRNAs are contemplated. Any reliable, sensitive, and specific method can be used. In some embodiments, a miRNA is amplified prior to measurement. In other embodiments, the level of miRNA is measured during the amplification process. In still other methods, the miRNA is not amplified prior to measurement.

Many methods exist for amplifying miRNA nucleic acid sequences such as mature miRNAs, precursor miRNAs, and primary miRNAs. Suitable nucleic acid polymerization and amplification techniques include reverse transcription (RT), polymerase chain reaction (PCR), real-time PCR (quantitative PCR (q-PCR)), nucleic acid sequence-base amplification (NASBA), ligase chain reaction, multiplex ligatable probe amplification, invader technology (Third Wave), rolling circle amplification, in vitro transcription (IVT), strand displacement amplification, transcription-mediated amplification (TMA), RNA (Eberwine) amplification, and other methods that are known to persons skilled in the art. In certain embodiments, more than one amplification method is used, such as reverse transcription followed by real time quantitative PCR (qRT-PCR) (Chen et al., Nucleic Acids Research, 33(20):e179 (2005)).

A typical PCR reaction includes multiple amplification steps, or cycles that selectively amplify target nucleic acid species: a denaturing step in which a target nucleic acid is denatured; an annealing step in which a set of PCR primers (forward and reverse primers) anneal to complementary DNA strands; and an elongation step in which a thermo stable DNA polymerase elongates the primers. By repeating these steps multiple times, a DNA fragment is amplified to produce an amplicon, corresponding to the target DNA sequence. Typical PCR reactions include 20 or more cycles of denaturation, annealing, and elongation. In many cases, the annealing and elongation steps can be performed concurrently, in which case the cycle contains only two steps. Since mature miRNAs are single-stranded, a reverse transcription reaction (which produces a complementary cDNA sequence) may be performed prior to PCR reactions. Reverse transcription reactions include the use of, e.g., a RNA-based DNA polymerase (reverse transcriptase) and a primer.

In PCR and q-PCR methods, for example, a set of primers is used for each target sequence. In certain embodiments, the lengths of the primers depends on many factors, including, but not limited to, the desired hybridization temperature between the primers, the target nucleic acid sequence, and the complexity of the different target nucleic acid sequences to be amplified. In certain embodiments, a primer is about 15 to about 35 nucleotides in length. In other embodiments, a primer is equal to or fewer than 15, 20, 25, 30, or 35 nucleotides in length. In additional embodiments, a primer is at least 35 nucleotides in length.

In a further aspect, a forward primer can comprise at least one sequence that anneals to a miRNA biomarker and alternatively can comprise an additional 5′ non-complementary region. In another aspect, a reverse primer can be designed to anneal to the complement of a reverse transcribed miRNA. The reverse primer may be independent of the miRNA biomarker sequence, and multiple miRNA biomarkers may be amplified using the same reverse primer. Alternatively, a reverse primer may be specific for a miRNA biomarker.

In some embodiments, two or more miRNAs are amplified in a single reaction volume. One aspect includes multiplex q-PCR, such as qRT-PCR, which enables simultaneous amplification and quantification of at least two miRNAs of interest in one reaction volume by using more than one pair of primers and/or more than one probe. The primer pairs comprise at least one amplification primer that uniquely binds each miRNA, and the probes are labeled such that they are distinguishable from one another, thus allowing simultaneous quantification of multiple miRNAs. Multiplex qRT-PCR has research and diagnostic uses, including but not limited to detection of miRNAs for diagnostic, prognostic, and therapeutic applications.

The qRT-PCR reaction may further be combined with the reverse transcription reaction by including both a reverse transcriptase and a DNA-based thermostable DNA polymerase. When two polymerases are used, a “hot start” approach may be used to maximize assay performance (U.S. Pat. Nos. 5,411,876 and 5,985,619). For example, the components for a reverse transcriptase reaction and a PCR reaction may be sequestered using one or more thermoactivation methods or chemical alteration to improve polymerization efficiency (U.S. Pat. Nos. 5,550,044, 5,413,924, and 6,403,341).

In certain embodiments, labels, dyes, or labeled probes and/or primers are used to detect amplified or unamplified miRNAs. The skilled artisan will recognize which detection methods are appropriate based on the sensitivity of the detection method and the abundance of the target. Depending on the sensitivity of the detection method and the abundance of the target, amplification may or may not be required prior to detection. One skilled in the art will recognize the detection methods where miRNA amplification is preferred.

A probe or primer may include Watson-Crick bases or modified bases. Modified bases include, but are not limited to, the AEGIS bases (from Eragen Biosciences), which have been described, e.g., in U.S. Pat. Nos. 5,432,272, 5,965,364, and 6,001,983. In certain aspects, bases are joined by a natural phosphodiester bond or a different chemical linkage. Different chemical linkages include, but are not limited to, a peptide bond or a Locked Nucleic Acid (LNA) linkage, which is described, e.g., in U.S. Pat. No. 7,060,809.

In a further aspect, oligonucleotide probes or primers present in an amplification reaction are suitable for monitoring the amount of amplification product produced as a function of time. In certain aspects, probes having different single stranded versus double stranded character are used to detect the nucleic acid. Probes include, but are not limited to, the 5′-exonuclease assay (e.g., TaqMan™) probes (see U.S. Pat. No. 5,538,848), stem-loop molecular beacons (see, e.g., U.S. Pat. Nos. 6,103,476 and 5,925,517), stemless or linear beacons (see, e.g., WO 9921881, U.S. Pat. Nos. 6,485,901 and 6,649,349), peptide nucleic acid (PNA) Molecular Beacons (see, e.g., U.S. Pat. Nos. 6,355,421 and 6,593,091), linear PNA beacons (see, e.g. U.S. Pat. No. 6,329,144), non-FRET probes (see, e.g., U.S. Pat. No. 6,150,097), Sunrise™/AmplifluorB™ probes (see, e.g., U.S. Pat. No. 6,548,250), stem-loop and duplex Scorpion™ probes (see, e.g., U.S. Pat. No. 6,589,743), bulge loop probes (see, e.g., U.S. Pat. No. 6,590,091), pseudo knot probes (see, e.g., U.S. Pat. No. 6,548,250), cyclicons (see, e.g., U.S. Pat. No. 6,383,752), MGB Eclipse™ probe (Epoch Biosciences), hairpin probes (see, e.g., U.S. Pat. No. 6,596,490), PNA light-up probes, antiprimer quench probes (Li et al., Clin. Chem. 53:624-633 (2006)), self-assembled nanoparticle probes, and ferrocene-modified probes described, for example, in U.S. Pat. No. 6,485,901.

In certain embodiments, one or more of the primers in an amplification reaction can include a label. In yet further embodiments, different probes or primers comprise detectable labels that are distinguishable from one another. In some embodiments a nucleic acid, such as the probe or primer, may be labeled with two or more distinguishable labels.

In some aspects, a label is attached to one or more probes and has one or more of the following properties: (i) provides a detectable signal; (ii) interacts with a second label to modify the detectable signal provided by the second label, e.g., FRET (Fluorescent Resonance Energy Transfer); (iii) stabilizes hybridization, e.g., duplex formation; and (iv) provides a member of a binding complex or affinity set, e.g., affinity, antibody-antigen, ionic complexes, hapten-ligand (e.g., biotin-avidin). In still other aspects, use of labels can be accomplished using any one of a large number of known techniques employing known labels, linkages, linking groups, reagents, reaction conditions, and analysis and purification methods.

MiRNAs can be detected by direct or indirect methods. In a direct detection method, one or more miRNAs are detected by a detectable label that is linked to a nucleic acid molecule. In such methods, the miRNAs may be labeled prior to binding to the probe. Therefore, binding is detected by screening for the labeled miRNA that is bound to the probe. The probe is optionally linked to a bead in the reaction volume.

In certain embodiments, nucleic acids are detected by direct binding with a labeled probe, and the probe is subsequently detected. In one embodiment of the invention, the nucleic acids, such as amplified miRNAs, are detected using FIexMAP Microspheres (Luminex) conjugated with probes to capture the desired nucleic acids. Some methods may involve detection with polynucleotide probes modified with fluorescent labels or branched DNA (bDNA) detection, for example.

In other embodiments, nucleic acids are detected by indirect detection methods. For example, a biotinylated probe may be combined with a streptavidin-conjugated dye to detect the bound nucleic acid. The streptavidin molecule binds a biotin label on amplified miRNA, and the bound miRNA is detected by detecting the dye molecule attached to the streptavidin molecule. In one embodiment, the streptavidin-conjugated dye molecule comprises Phycolink® Streptavidin R-Phycoerythrin (PROzyme). Other conjugated dye molecules are known to persons skilled in the art.

Labels include, but are not limited to: light-emitting, light-scattering, and light-absorbing compounds which generate or quench a detectable fluorescent, chemiluminescent, or bioluminescent signal (see, e.g., Kricka, L., Nonisotopic DNA Probe Techniques, Academic Press, San Diego (1992) and Garman A., Non-Radioactive Labeling, Academic Press (1997).). Fluorescent reporter dyes useful as labels include, but are not limited to, fluoresceins (see, e.g., U.S. Pat. Nos. 5,188,934, 6,008,379, and 6,020,481), rhodamines (see, e.g., U.S. Pat. Nos. 5,366,860, 5,847,162, 5,936,087, 6,051,719, and 6,191,278), benzophenoxazines (see, e.g., U.S. Pat. No. 6,140,500), energy-transfer fluorescent dyes, comprising pairs of donors and acceptors (see, e.g., U.S. Pat. Nos. 5,863,727; 5,800,996; and 5,945,526), and cyanines (see, e.g., WO 9745539), lissamine, phycoerythrin, Cy2, Cy3, Cy3.5, Cy5, Cy5.5, Cy7, FluorX (Amersham), Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, Tetramethylrhodamine, and/or Texas Red, as well as any other fluorescent moiety capable of generating a detectable signal. Examples of fluorescein dyes include, but are not limited to, 6-carboxyfluorescein; 2′,4′, 1,4,-tetrachlorofluorescein; and 2′,4′, 5′,7′, 1,4-hexachlorofluorescein. In certain aspects, the fluorescent label is selected from SYBR-Green, 6-carboxyfluorescein (“FAM”), TET, ROX, VIC™, and JOE. For example, in certain embodiments, labels are different fluorophores capable of emitting light at different, spectrally-resolvable wavelengths (e.g., 4-differently colored fluorophores); certain such labeled probes are known in the art and described above, and in U.S. Pat. No. 6,140,054. A dual labeled fluorescent probe that includes a reporter fluorophore and a quencher fluorophore is used in some embodiments. It will be appreciated that pairs of fluorophores are chosen that have distinct emission spectra so that they can be easily distinguished.

In still a further aspect, labels are hybridization-stabilizing moieties which serve to enhance, stabilize, or influence hybridization of duplexes, e.g., intercalators and intercalating dyes (including, but not limited to, ethidium bromide and SYBR-Green), minor-groove binders, and cross-linking functional groups (see, e.g., Blackburn et al., eds. “DNA and RNA Structure” in Nucleic Acids in Chemistry and Biology (1996)).

In further aspects, methods relying on hybridization and/or ligation to quantify miRNAs may be used, including oligonucleotide ligation (OLA) methods and methods that allow a distinguishable probe that hybridizes to the target nucleic acid sequence to be separated from an unbound probe. As an example, HARP-like probes, as disclosed in U.S. Publication No. 2006/0078894 may be used to measure the quantity of miRNAs. In such methods, after hybridization between a probe and the targeted nucleic acid, the probe is modified to distinguish the hybridized probe from the unhybridized probe. Thereafter, the probe may be amplified and/or detected. In general, a probe inactivation region comprises a subset of nucleotides within the target hybridization region of the probe. To reduce or prevent amplification or detection of a HARP probe that is not hybridized to its target nucleic acid, and thus allow detection of the target nucleic acid, a post-hybridization probe inactivation step is carried out using an agent which is able to distinguish between a HARP probe that is hybridized to its targeted nucleic acid sequence and the corresponding unhybridized HARP probe. The agent is able to inactivate or modify the unhybridized HARP probe such that it cannot be amplified.

In an additional embodiment of the method, a probe ligation reaction may be used to quantify miRNAs. In a Multiplex Ligation-dependent Probe Amplification (MLPA) technique (Schouten et al., Nucleic Acids Research 30:e57 (2002)), pairs of probes which hybridize immediately adjacent to each other on the target nucleic acid are ligated to each other only in the presence of the target nucleic acid. In some aspects, MLPA probes have flanking PCR primer binding sites. MLPA probes can only be amplified if they have been ligated, thus allowing for detection and quantification of miRNA biomarkers.

In a particular embodiment, miRNA lung cancer biomarkers are measured according to Shen et al. Lab Invest. (2011), wherein miRNA is purified from a serum sample using a mirVana miRNA isolation kit from Ambion followed by amplification and detection by RT-PCT, such as with a TaqMan MicroRNA RT kit from Applied Biosystems.

ii) Analysis of Biomarkers

Once measured, the measurement for each biomarker in a given panel is analyzed in aggregate to provide a probability of cancer. In certain embodiments, the probability or likelihood of cancer is represented as a percentage the tested patient is positive for the presence of lung cancer—their risk of having lung cancer.

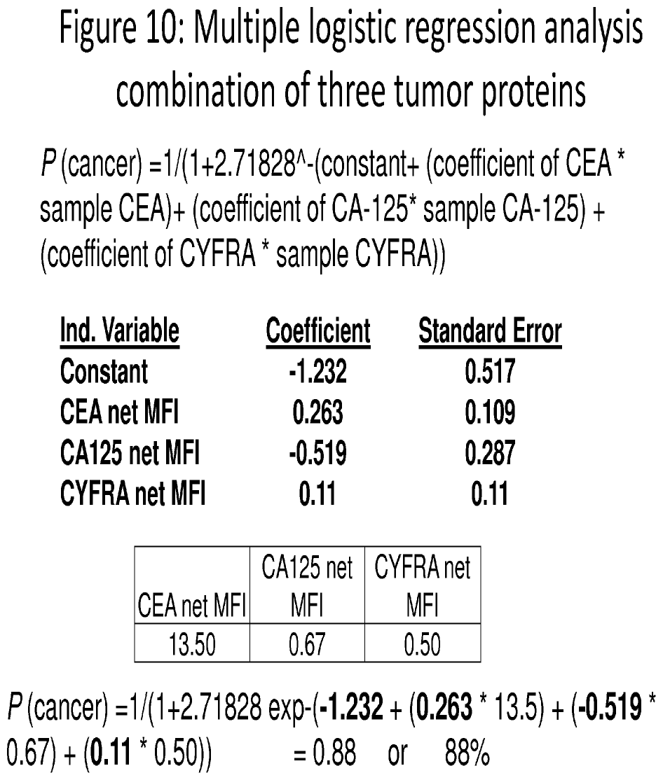

In certain embodiments the probability of cancer is calculated using standard statistical analysis well known to one of skill in the art wherein the measurements of each lung cancer biomarker in the panel are combined to provide a probability of cancer. In one aspect multiple logistic regression analysis is used to derive a mathematical function with a set of variables corresponding to each marker, which provides a weighting factor for each biomarker. The weighting factor are derived to optimize the agency of the function to predict the dependent variable, which in Examples 1 and 2 was the dichotomy of cancer or non-cancer in the patients. The weighting factors are specific to the particular biomarker combination (e.g. panel) analyzed. The function can then be applied to the original samples to predict a probability. See FIG. 10 as an example calculation for determining probability of cancer using multiple logistic regression analysis. In this way, a retrospective data set is used to provide weighting factors for a particular panel of lung cancer biomarkers, which is then used to calculated the probability of cancer in a patient where the outcome of cancer is unknown prior to screening using the present methods.

Other established methods may also be used to analyze the measurement data from the lung cancer biomarkers in a patient sample to either diagnose cancer and/or determine the likelihood a patient has cancer and/or determining risk a patient has cancer and/or determining the increase in risk of cancer to a patient.

The choice of the markers may be based on the understanding that each marker, when measured and normalized, contributed equally to determine the likelihood of the presence of the cancer. Thus in certain embodiments, the each marker in the panel is measured and normalized wherein none of the markers are given any specific weight. In this instance each marker has a weight of 1.

In other embodiments, the choice of the markers may be based on the understanding that each marker, when measured and normalized, contributed unequally to determine the likelihood of the presence of the cancer. In this instance, a particular marker in the panel can either be weighted as a fraction of 1 (for example if the relative contribution is low), a multiple of 1 (for example if the relative contribution is high) or as 1 (for example when the relative contribution is neutral compared to the other markers in the panel). Thus, in certain embodiments, the present methods further comprising weighting the normalized values prior to summation of the normalized values to obtain a composite score.

Decision tree is a data handling approach where a series of simple dichotomous decisions guide through a classification to yield such a desired binary outcome. Hence, samples are partitioned based on whether values thereof are above or below calculated thresholds.

A model for scoring multiple biomarkers which attempts to employ a decision tree logic was developed by Mor et al., PNAS, 102(21):7677-7682 (2005), wherein an optimal cutoff value is obtained and assigns a value of 0 (not likely to have cancer) or 1 (likely to have cancer) for a marker. Then, scores of individual biomarkers are combined for a final score of each sample and the higher the final score, the higher the probability of disease.

That technique provides a binary result favored by physicians and patients. While distribution of data is not an assumption which contributes to simplicity of the model, that the model reduces information to a 1 or 0 score results in a loss of quantitative information, for example, diminishes the role of a more predictive marker and elevates the role of a less predictive marker.

Moreover, the collection of markers in a multiplex assay may comprise varying levels of value or predictability in diagnosing disease. Hence, the impact of any one marker on the ultimate determination may be weighted based on the aggregated data obtained in screening populations and correlating with actual pathology to provide a more discriminating or effective diagnostic assay.

An alternative approach is to find an intermediate ground by expanding the qualitative transformation of quantitative data into multiple categories as compared to only a binary classification scheme.

In certain embodiments, the step of normalizing comprises determining the multiple of median (MoM) score for each marker. In this instance, the MoM score is the subsequently summed to obtained a composite score.

In other embodiments, obtaining a probability of cancer may further comprise normalizing the measured biomarker values and summing the normalized values to generate a probability of cancer.

In certain embodiments, the value obtained from measuring the marker in the sample is normalized. There is no intended limitation on the methodology used to normalize the values of the measured biomarkers.

Many methods for data normalization exist as are familiar to those skilled in the art. These include methods as simple as background subtraction, scaling, multiple of the median (MoM) analysis, linear transformation, least squares fitting, etc. The goal of normalization is to equate the varying measurement scales for the separate markers such that the resulting values may be combined according to a separate a weighting scale as determined and designed by the user and are not influenced by the absolute or relative values of the marker found within nature.

US Publ. No. 2008/0133141 (herein incorporated by reference) teaches statistical methodology for handling and interpreting data from a multiplex assay. The amount of any one marker thus can be compared to a predetermined cutoff distinguishing positive from negative for that marker as determined from a control population study of patients with cancer and suitably matched normal controls to yield a score for each marker based on said comparison; and then combining the scores for each marker to obtain a composite score for the marker(s) in the sample.

A predetermined cutoff can be based on ROC curves and the score for each marker can be calculated based on the specificity of the marker. Then, the total score can be compared to a predetermined total score to transform that total score to a qualitative determination of the likelihood or risk of having lung cancer.

Another method for score transformation or normalization is, for example, applying the multiple of median (MoM) method of data integration. In the MoM method, the median value of each biomarker is used to normalize all measurements of that specific biomarker, for example, as provided in Kutteh et al. (Obstet. Gynecol. 84:811-815, 1994) and Palomaki et al. (Clin. Chem. Lab. Med.) 39:1137-1145, 2001). Thus, any measured biomarker level is divided by the median value of the cancer group, resulting in a MoM value. The MoM values can be combined (namely, summed or added) for each biomarker in the panel resulting in a panel MoM value or aggregate MoM score for each sample.

In certain embodiments, the biomarkers are measured and those resulting values normalized and then summed to obtain a composite score. In certain aspects, normalizing the measured biomarker values comprises determining the multiple of median (MoM) score. In other aspects, the present method further comprises weighting the normalized values before summing to obtain a composite score.

Primary care healthcare practitioners, who may include physicians specializing in internal medicine or family practice as well as physician assistants and nurse practitioners, are among the users of the methodology disclosed herein. These primary care providers typically see a large volume of patients each day and many of these patients are at risk for lung cancer due to smoking history, age, and other lifestyle factors. In 2012 about 18% of the U.S. population was current smokers and many more were former smokers with a lung cancer risk profile above that of never smokers.

The aforementioned NLST study (See, background section) concluded that heavy smokers over a certain age who undergo yearly screening with CT scans have a substantial reduction in lung cancer mortality as compared to those who are not similarly screened. Nevertheless, for the reasons discussed above, very few at risk patients are undergoing annual CT screening. For these patients the testing paradigm according to the present invention offers an alternative.

A blood sample from patients with a heavy smoking history (e.g. having smoked at least a pack of cigarettes per day for 20 years or more) is sent to a laboratory qualified to test the sample using a panel of biomarkers with adequate sensitivity and specific for early stage lung cancer. Non limiting lists of such biomarkers are herein included in the above disclosure and the following examples. In lieu of blood, other suitable bodily fluids such a sputum or saliva might also be utilized.

A probability of cancer for that patient is then generated using the technique described in the present disclosure. Using the probability of cancer value the patient's risk of having lung cancer, as compared to others having a comparable smoking history and age range, can then be calculated. In particular, if the risk calculation is to be made at the point of care, rather than at the laboratory, a software application compatible with mobile devices (e.g. a tablet or smart phone) may be employed.

Once the physician or healthcare practitioner has a risk score for the patient (i.e. the likelihood that that patient has lung cancer relative to a population of others with comparable epidemiological factors) they can recommend, in particular, that those at a higher risk be followed up with other tests such as CT scanning. It should be appreciated that the precise numerical cut off above which further testing is recommended may vary depending on many factors including, without limitation, (i) the desires of the patients and their overall health and family history, (ii) practice guidelines established by medical boards or recommended by scientific organizations, (iii) the physician's own practice preferences, and (iv) the nature of the biomarker test including its overall accuracy and strength of validation data.

It is believed that use of the methodology disclosed herein will have the twin benefits of ensuring that the most at risk patients undergo CT scanning so as to detect early tumors that can be cured with surgery while reducing the expense and burden of false positives associated with stand-alone CT screening.

All references cited herein are herein incorporated by reference in entirety.

### EXAMPLES

The Examples below are given so as to illustrate the practice of this invention. They are not intended to limit or define the entire scope of this invention.

### Example 1: Study of Lung Cancer Biomarker Expression in Retrospective Clinical Samples

A cohort of 24 cases of stage Ia and Ib lung cancer, consisting of 13 adenocarcinomas and 11 squamous cell carcinomas, and 26 matched controls with benign lung lesions was obtained from the Veterans Administration. The associated patient information comprised the ages, genders, races, final diagnoses, histological types, stages, and possibly the cigarette usage intensity in smoking-package years is present in Table 1.

TABLE 1
50 Samples tested with lung cancer biomarker panels.

| | | | | final | | | smoking- |
|---|---|---|---|---|---|---|---|
| Patient ID# | ages | gender | race | diagnosis | histological types | stage | pack years |
| VA222 | 76 | M | W | NSCLC | Squamous cell CA | Ia | 65 |
| VA183 | 61 | M | AA | NSCLC | Adenocarcinoma | I | 30 |
| VA170 | 71 | M | AA | NSCLC | Adenocarcinoma | Ia | 45 |
| VA159 | 62 | M | AA | NSCLC | Adenocarcinoma | Ia | 30 |
| VA189 | 67 | M | W | NSCLC | Adenocarcinoma | Ia | 50 |
| VA217 | 76 | M | AA | NSCLC | Adenocarcinoma | Ib | 250 |
| VA254 | 73 | M | W | NSCLC | Adenocarcinoma | I | 81 |
| VA282 | 73 | M | W | NSCLC | Adenocarcinoma | Ib | 60 |
| VA243 | 64 | M | AA | NSCLC | Squamous cell CA | I | 50 |
| VA391 | 67 | M | W | NSCLC | Squamous Cell CA | Ib | 200 |
| VA319 | 69 | M | AA | NSCLC | Adenocarcinoma | Ia | 50 |
| UM413 | 64 | M | AA | NSCLC | Adenocarcinoma | Ib | 20 |
| VA380 | 66 | M | W | NSCLC | Adenocarcinoma | Ia | 45 |
| VA449 | 74 | M | W | NSCLC | Adenocarcinoma | Ia | 10 |
| VA190 | 66 | M | W | NSCLC | Adenocarcinoma | Ia | 125 |
| VA172 | 59 | M | W | NSCLC | Adenocarcinoma | Ia | 4 |
| VA369 | 73 | M | W | NSCLC | Squamous Cell CA | Ia | 120 |
| VA236 | 67 | M | W | NSCLC | Squamous cell CA | Ia | 100 |
| VA423 | 61 | M | AA | NSCLC | Squamous cell CA | Ia | 20 |
| VA473 | 68 | M | W | NSCLC | Squamous cell CA | Ib | 50 |
| VA547 | 77 | M | AA | NSCLC | Squamous cell CA | Ia | 60 |
| VA428 | 87 | M | W | NSCLC | Squamous cell CA | Ia | 37 |
| VA352 | 71 | M | AA | NSCLC | Squamous Cell CA | Ia | 25 |
| VA277 | 61 | M | AA | NSCLC | Squamous cell CA | Ia | 50 |
| UM331 | 74 | M | W | Benign | | | 0 |
| VA412 | 51 | M | AA | Benign | | | 0 |
| VA437 | 64 | M | W | Benign | | | 0 |
| VA522 | 63 | M | W | Benign | | | 0 |
| VA377 | 82 | M | AA | Benign | | | |
| VA513 | 46 | M | W | Benign | | | 0 |
| VA500 | 42 | M | W | Benign | | | 0 |
| VA278 | 62 | M | AA | Benign | | | 0 |
| VA264 | 79 | M | AA | Benign | Necrotizing | | 0 |
| | | | | | granulomas | | |
| VA307 | 84 | M | AA | Benign | Probable Sarcoid | | 0 |
| UM351 | 57 | M | W | Benign | | | 0 |
| VA523 | 28 | M | AA | Benign | | | 0 |
| VA534 | 66 | M | AA | Benign | | | 0 |
| VA365 | 65 | M | AA | Benign | | | 40 |
| VA413 | 61 | M | AA | Benign | | | 60 |
| VA389 | 77 | M | W | Benign | | | 30 |
| VA324 | 67 | M | W | Benign | | | 90 |
| VA402 | 74 | M | W | Benign | | | 120 |
| VA221 | 63 | M | W | Benign | Benign Nodule | | 105 |
| VA454 | 71 | M | W | Benign | | | 125 |
| VA537 | 52 | M | W | Benign | Reactive lymphoid | | 20 |
| | | | | | tissue | | |
| VA531 | 53 | M | W | Benign | | | 18.5 |
| VA421 | 76 | M | AA | Benign | | | 30 |
| VA364 | 58 | M | AA | Benign | | | 15 |
| VA157 | 60 | M | AA | Benign | Benign Nodule | | 90 |
| VA228 | 42 | M | W | Benign | | | 10 |

A multiplex diagnostic platform is an automated comprehensive system capable of isolating the target analyte (protein antigen or autoantibody), performing the test, and displaying the interpretation of the multiplex test result. To accomplish our multiplexed test we use a flow cytometry bead-based approach. Multiplex bead array assays provide quantitative measurement of large numbers of analytes using an automated 96-well plate format. The Luminex method uses microsphere sets carrying variable quantities of two different fluorescent dyes that produce up to 100 different shades of color. Each bead is coupled to a unique antibody or protein that recognizes a specific molecule. After the beads are mixed with a serum sample and added to the instrument, the unique color signature on each bead reveals the identity of the bound molecules. The level of fluorescence (reported as Median Flourescence Intensity or MFI) of the tagged antibody or protein indicates the level of antibody or protein in the serum.

The TP and AAB biomarkers included 3 autoantibody (AAB) biomarkers (p53 (Pierce RP-39232), NY-ESO-1(Pierce RP-39227), and Mapkapk3 (Genway 10-782-55070)) and 3 tumor protein (TP) biomarkers (CA125, CEA and CYFRA 21-1). These three AAB biomarkers as well as the TP CEA marker (anti-CEA, Abcam ab4451) are produced in-house using the Luminex beads/plateform technology. Commercially available reagents for CA125 and Cyfra 21-1 (Millipore HCCBP1MAG-58K-02) are used.

Autoantibody Assay

In this assay, protein (antigen) is coupled to Luminex beads. The beads (with 3 unique color signatures each with a single biomarker protein) are then incubated with the patient serum (capture of the specific autoantibody). After incubation and washing steps the bead/antibody complex is exposed to the fluorescent labeled anti-human reporter antibody (Thermo, PAI-86078). The complex is then washed again and then placed in the Luminex instrument. The color signature distinguishes the biomarker being measured and the median fluorescence intensity of the reporter indicates the amount of the autoantibody of interest. NY-ESO-1 is coupled to Luminex bead, region 35 (Luminex, MC10035), p53 is coupled to Luminex bead, region 43 (Luminex, MC10043) and MapkapK3 is coupled to Luminex bead, region 45 (Luminex, MC10043)

Tumor Protein Assay

In this assay an antibody to the protein of interest is coupled to a surface-Luminex bead. The bead is then incubated with the patient serum. The protein of interest binds to the antibody coated bead (capture). Next, a second antibody (detection) is incubated with the capture antibody-protein complex. The detection antibody is labeled with a fluorescent tag. After washing unbound material away, the complex or “sandwich” (capture antibody-protein-detection antibody) is placed in the Luminex instrument. The color signature of the Luminex bead indicates the analyte being measured and the Median Flourescent Intensity (MFI) measures the amount of protein biomarker present in the sample.

The two assays have different incubation times etc., so for this reason two separate multiplex assays are performed. The raw data for each biomarker assay consisted of a median fluorescence intensity (MFI), measured in triplicate, minus a blank measured in triplicate

miRNA Assay

For this assay, the miRNA (miRNA21, miRNA126, miRNA210 and miRNA486) was purified and amplified according to protocols described in Shen et al. Lab Invest. (2011). Briefly, miRNA was purified from a serum sample by using a mirVana miRNA isolation Kit (Ambion). The miRNA was subjected to analysis on a Bioanalyzer 2100 (Agilent), and was accepted only if the integrity number was above 6. Reverse transcription (RT)-qPCR was done with a TaqMan MicroRNA RT Kit (Applied Biosystems). The raw data quantitative PCR data consisted of threshold cycle number (CO of reverse transcribed, real time PCR (RT-qPCR). miRNA biomarker values were normalized to an internal control miRNA which was miR-16. The fold change (or expression ratio) was calculated using the equation 2^{-ΔΔCt}.

Analysis

In SigmaPlot 12.5, the pre-processed raw data for all cases were subjected to automated ROC analysis. The output included a table of AUC values, sensitivities and specificities corresponding to ordered series of cutoff points. See FIGS. 1-3.

The ten individual lung cancer biomarkers from three groups (miRNA, TP and AAB) were designated as Control Group 1, Control Group 2 and Control Group 3 respectively. The lung cancer biomarkers were further grouped to form Control Group 4 (TP and AAB) and three Test Groups; Test Group 5 (miRNA and AAB), Test Group 6 (miRNA TP) and Test Group 7 (miRNA, TP and AAB). See FIGS. 4-9.

The diagnostic accuracy for classifying cancer or non-cancer was evaluated for three types of biomarkers, in sub-groups or combined with the miRNA biomarker group. The analysis demonstrated an increased sensitivity (at 80% specificity) for all Test Groups (combination with miRNA biomarkers) and an AUC of at least 0.89. The ranked order of accuracy was Test Group 7 (miRNA, TP and AAB)>Test Group 6 (miRNA TP)>Test Group 5 (miRNA and AAB)>Control Group 4 (TP and AAB)>Control Group 1 (miRNA)>Control Group 2 (TP)>Control Group 3 (AAB). Diagnostic accuracy, as used herein, refers to the average of the sensitivity and specificity.

### Example 2: Patient Test Results and Validation

The data from each of the 7 groups were also analyzed at the individual patient level to provide a probability of cancer (as a percentage) for each patient using the raw data from the measurement of biomarkers (data not shown). See Table 2 below. The cutoff value was based on an 80% specificity for each of the seven panels and ranges from 15% to 50% depending on the panel. This analysis validates the use of each panel for determining the likelihood of cancer for a patient and demonstrates the improvement in the lung cancer biomarker panels comprising at least one miRNA lung cancer biomarker.

The biomarker data was combined in a standard statistical analysis method well known in the art for determining the probability of cancer for an individual patient. Multiple Logistic Regression analysis was used to derive a mathematical function with a set of variables corresponding to each biomarker, which provides a weighting factor for each marker. The weighting factors were derived to optimize the agency of the function to predict the dependent variable, which was the dichotomy of cancer or non-cancer in the patients. The weighting factors were specific to the particular biomarker combinations analyzed. The function was then applied to the original samples to predict a probability. See FIG. 10.

The shaded Patient ID# are those patients previously diagnosed with stage I lung cancer and those Patient ID# not shaded correspond to patients with benign lung lesions. See Table 1 for more patient detail. In Table 2, for the cancer group, the individual shaded boxes represent true positives and the unshaded boxes represent false negatives. For the non-cancer group, the shaded boxes represent false positives and the unshaded boxes represent true negatives. The biomarker panels of Test Group 6 and 7 correctly identified all but one of the patients in the cancer group. The number of false positives were about the same (4-6) for all groups because the specificity was set at about 80% (81%-85%) for all biomarker panel groups, this allowed for a clear demonstration of the improvement in identifying the true positives in the sample set with each biomarker panel group.

TABLE 2
Patient Data Set from Table 1 with corresponding probability
of cancer for each of the seven panels tested.

| | Biomarker Panels | | | | | | |
|---|---|---|---|---|---|---|---|
| | Test | Test | Test | Control | Control | Control | Control |
| Patient ID# | Group 7 | Group 6 | Group 5 | Group 1 | Group 4 | Group 2 | Group 3 |
| VA222 | 99% | 80% | 52% | 20% | 98% | 88% | 67% |
| VA183 | 29% | 22% | 52% | 42% | 36% | 28% | 53% |
| VA170 | 91% | 68% | 47% | 56% | 82% | 58% | 46% |
| VA159 | 78% | 72% | 71% | 92% | 20% | 30% | 35% |
| VA189 | 45% | 66% | 25% | 55% | 41% | 70% | 29% |
| VA217 | 100% | 100% | 73% | 98% | 100% | 100% | 40% |
| VA254 | 3% | 12% | 11% | 25% | 31% | 41% | 36% |
| VA282 | 100% | 100% | 95% | 100% | 98% | 98% | 38% |
| VA243 | 100% | 99% | 97% | 100% | 59% | 58% | 39% |
| VA391 | 99% | 97% | 90% | 99% | 82% | 64% | 42% |
| VA319 | 96% | 86% | 18% | 25% | 100% | 96% | 34% |
| UM413 | 100% | 100% | 71% | 98% | 100% | 100% | 30% |
| VA380 | 92% | 97% | 93% | 100% | 16% | 29% | 31% |
| VA449 | 90% | 77% | 60% | 94% | 80% | 57% | 48% |
| VA190 | 85% | 24% | 86% | 56% | 63% | 27% | 71% |
| VA172 | 40% | 45% | 71% | 91% | 23% | 26% | 41% |
| VA369 | 81% | 54% | 88% | 98% | 46% | 21% | 55% |
| VA236 | 85% | 86% | 84% | 99% | 38% | 39% | 43% |
| VA423 | 100% | 85% | 93% | 64% | 98% | 75% | 68% |
| VA473 | 100% | 100% | 69% | 99% | 100% | 98% | 31% |
| VA547 | 100% | 97% | 100% | 90% | 100% | 93% | 98% |
| VA428 | 44% | 70% | 76% | 99% | 7% | 20% | 36% |
| VA352 | 59% | 50% | 85% | 96% | 67% | 41% | 59% |
| VA277 | 54% | 78% | 79% | 99% | 31% | 43% | 31% |
| UM331 | 3% | 6% | 1% | 2% | 24% | 27% | 34% |
| VA412 | 0% | 0% | 6% | 1% | 8% | 10% | 36% |
| VA437 | 10% | 8% | 7% | 4% | 36% | 29% | 36% |
| VA522 | 13% | 9% | 28% | 18% | 20% | 18% | 35% |
| VA377 | 4% | 20% | 3% | 9% | 18% | 35% | 33% |
| VA513 | 18% | 47% | 15% | 45% | 11% | 31% | 31% |
| VA500 | 3% | 10% | 10% | 10% | 14% | 25% | 34% |
| VA278 | 0% | 0% | 8% | 4% | 0% | 0% | 37% |
| VA264 | 6% | 8% | 7% | 4% | 39% | 36% | 43% |
| VA307 | 1% | 2% | 3% | 2% | 23% | 28% | 35% |
| UM351 | 44% | 35% | 49% | 62% | 38% | 27% | 48% |
| VA523 | 13% | 21% | 43% | 61% | 16% | 20% | 34% |
| VA534 | 44% | 18% | 28% | 5% | 70% | 51% | 43% |
| VA365 | 0% | 0% | 1% | 1% | 6% | 8% | 38% |
| VA413 | 6% | 8% | 46% | 70% | 22% | 15% | 43% |
| VA389 | 1% | 0% | 16% | 1% | 25% | 8% | 64% |
| VA324 | 0% | 3% | 11% | 4% | 12% | 23% | 54% |
| VA402 | 2% | 2% | 37% | 19% | 10% | 8% | 50% |
| VA221 | 3% | 14% | 2% | 3% | 32% | 45% | 34% |
| VA454 | 1% | 11% | 2% | 4% | 23% | 38% | 36% |
| VA537 | 0% | 0% | 33% | [symbol] % | 6% | 9% | 37% |
| VA531 | 16% | 75% | 15% | 28% | 36% | 87% | 22% |
| VA421 | 0% | 1% | 27% | 44% | 2% | 8% | 33% |
| VA364 | 38% | 25% | 11% | 4% | 76% | 73% | 52% |
| VA157 | 0% | 0% | 4% | 15% | 1% | 4% | 27% |
| VA228 | 1% | 11% | 2% | 9% | 18% | 39% | 31% |
| ACCURACY | | | | | | | |
| ANALYSIS | | | | | | | |
| cutoff | >15.% | >21.% | >50.% | >45.% | >35.% | >39.% | >45.% |
| sensitivity | 96% | 96% | 88% | 83% | 71% | 71% | 38% |
| specificity | 81% | 81% | 81% | 81% | 85% | 81% | 81% |
| average accuracy | 88% | 88% | 84% | 82% | 78% | 76% | 59% |

What is claimed is:

1. A method of assessing the likelihood that a patient has lung cancer, comprising:
measuring a level of at least one microRNA lung cancer biomarker in a sample from the human subject; and
measuring a level of at least one cancer biomarker selected from a tumor protein lung cancer biomarker or an autoantibody lung cancer biomarker in a sample from the human subject;
calculating a probability of cancer from said biomarker measurements, whereby the likelihood that a patient has lung cancer is determined.

2. The method of claim 1, wherein the at least one cancer biomarker is a tumor protein lung cancer biomarker.

3. The method of claim 1, wherein the at least one cancer biomarker is an autoantibody lung cancer biomarker.

4. The method of claim 1, further comprising measuring at least one tumor protein lung cancer biomarker and at least one autoantibody lung cancer biomarker.

5. The method of claim 1, wherein the at least one cancer biomarker is selected from the group consisting of CEA, CA125, Cyfra 21-1, Pro-GRP, anti-NY-ESO-1, anti-p53, anti-Cyclin E2 and anti-MAPKAPK3.

6. The method of claim 1, wherein the at least one microRNA lung cancer biomarker is Mir21, Mir126, Mir210 or Mir486.

7. The method of claim 1, wherein the patient is over age 50 and has a history of smoking cigarettes.

8. The method of claim 7, wherein the smoking history comprises at least about a 20 pack year smoking history.

9. The method of claim 1, wherein the sample is blood, blood serum, blood plasma, or some part thereof.

10. A method for predicting a patient is positive for lung cancer, comprising:
measuring levels of lung cancer biomarkers comprising at least one microRNA lung cancer biomarker and at least one tumor protein lung cancer biomarker and/or at least one autoantibody lung cancer biomarker in a sample from the human subject;
calculating a probability value for cancer from the measured lung cancer biomarker levels;
comparing the probability value to a threshold value to determine whether or not it is above or below the threshold value;
concluding, if the probability value is above the threshold value, that the patient is positive for lung cancer, or
concluding, if the probability value is below the threshold value, that the patient is negative for lung cancer.

11. The method of claim 10, wherein the threshold value is a sensitivity value for the measured lung cancer biomarkers.

12. The method of claim 11, wherein the sensitivity value is calculated based on a cut off of about 70% specificity or greater.

13. The method of claim 11, wherein the sensitivity value is calculated based on a cut off of about 80% specificity or greater.

14. The method of claim 10, wherein the at least one microRNA lung cancer biomarker is Mir21, Mir126, Mir210 or Mir486.

15. The method of claim 10, wherein the measuring includes measuring a panel of lung cancer biomarkers comprising at least one microRNA lung cancer biomarker, at least one tumor protein lung cancer biomarker and at least one autoantibody lung cancer biomarker.

16. The method of claim 15, wherein the at least one microRNA lung cancer biomarker is Mir21, Mir126, Mir210 or Mir486; the at least one tumor protein lung cancer biomarker is CEA, CA125, Cyfra 21-1, Pro-GRP; and the at least one autoantibody lung cancer biomarker is anti-NY-ESO-1, anti-p53, anti-Cyclin E2 and anti-MAPKAPK3.

17. A method for increasing sensitivity of diagnosing lung cancer in a patient, comprising:
measuring levels of lung cancer biomarkers comprising at least one microRNA lung cancer biomarker and at least one tumor protein lung cancer biomarker and/or at least one autoantibody lung cancer biomarker in a sample from the human subject;
calculating a sensitivity value for the measured lung cancer biomarkers, wherein the sensitivity is increased as compared to a sensitivity value calculated by measuring lung cancer biomarkers without at least one microRNA lung cancer biomarker.

18. The method of claim 17, wherein the at least one microRNA lung cancer biomarker is Mir21, Mir126, Mir210 or Mir486.

19. The method of claim 17, further comprising measuring at least one tumor protein lung cancer biomarker and at least one autoantibody lung cancer biomarker.

20. The method of claim 17, wherein the at least one microRNA lung cancer biomarker is Mir21, Mir126, Mir210 or Mir486; the at least one tumor protein lung cancer biomarker is CEA, CA125, Cyfra 21-1, Pro-GRP; and the at least one autoantibody lung cancer biomarker is anti-NY-ESO-1, anti-p53, anti-Cyclin E2 and anti-MAPKAPK3.
